SEQUENCE LISTING (11 sequences)

SEQ ID NO: 1 - DNA, 21 bp, Homo sapiens
aaagaaatttatggccctgag

SEQ ID NO: 2 - DNA, 22 bp, Homo sapiens
ctagctttctgccattctcatc

SEQ ID NO: 3 - DNA, 22 bp, Homo sapiens
gaagagctggtcaatctcagaa

SEQ ID NO: 4 - DNA, 29 bp, Homo sapiens
aacccgaattctgtccgacagcgagaagc

SEQ ID NO: 5 - DNA, 31 bp, Homo sapiens
aaggactcgagctatttcttggctttggcgg

SEQ ID NO: 6 - DNA, 23 bp, Homo sapiens
aaaataaaggctctacaggaagc

SEQ ID NO: 7 - DNA, 23 bp, Homo sapiens
gcttcctgtagagcctttatttt

SEQ ID NO: 8 - DNA, 20 bp, Homo sapiens
gcatgactggtaccatggtg

SEQ ID NO: 9 - DNA, 22 bp, Homo sapiens
ttcttgctctcgagatacaaca

SEQ ID NO: 10 - DNA, 18 bp, Homo sapiens
tttctgaccgccctgagg

SEQ ID NO: 11 - DNA, 18 bp, Homo sapiens
cctcagggcggtcagaaa

(19) United States
(12) Patent Application Publication
(10) Pub. No.: US 2010/0151452 A1
(43) Pub. Date: Jun. 17, 2010

(54) Methods and Systems for Searching for Regulators of the Fer Protein and for Monitoring the Effects of the Fer Protein

(76) Inventors: Uri Nir, Moshav Gamzo (IL); Orel Pasder, Gedera (IL); Sally Shpungin, Ramat-gan (IL); Etai Yaffe, Oranit (IL)

Correspondence Address: THE NATH LAW GROUP, 112 South West Street, Alexandria, VA, 22314, US

(21) Appl. No.: 12/225,238

(22) PCT Filed: Mar. 20, 2007
(86) PCT No.: PCT/IL2007/000360

§ 371 Date: Dec. 8, 2008

Related U.S. Application Data
(60) Provisional application No. 60/783,390, filed on Mar. 20, 2006.

(51) Int. Cl. C12Q 1/68

(52) U.S. Cl. 435/6

(57) ABSTRACT

A method for modulating a Fer mediated pathway in a cell, for monitoring the effect of a Fer protein in a cell, for altering and inhibiting one or more effects of a Fer protein in a cell is provided. The method includes modulating, monitoring, altering or inhibiting any one or more or of the association of the Fer protein to PP1; the phosphorylation of PP1, the phosphatase activity of PP1, and the tumor suppressive activity of the retinoblastoma protein (RB). In a preferred embodiment, modulating a Fer mediated pathway in a cell, monitoring the effect of a Fer protein in a cell, for altering and inhibiting one or more effects of a Fer protein in a cell involves administrating to a cell a compound having a molecular weight of up to 1000 Dalton capable of modulating, altering or inhibiting an effect of Fer. Further provided is a method for determining whether a substance is capable of altering or inhibiting an effect of a Fer protein in a cell. The method includes presenting the substance to a first cell expressing an exogenous Fer gene or Fer cDNA; and measuring a rate of proliferation of the cell. A significant difference between the growth rate of the first cell and the growth rate of a second cell not expressing an exogenous Fer gene or Fer cDNA indicates that the substance is capable of altering a fer mediated pathway.

### FIELD OF THE INVENTION

This invention relates to methods and systems cellular and molecular biology, and more specifically to such systems and methods for searching for regulators of a protein and for monitoring the activity of a protein.

### BACKGROUND OF THE INVENTION

The following references are considered to be relevant for an understanding of the invention.

### REFERENCES

U.S. patent application Ser. No. 10/486,101, having the Publication Number 20050063973.
Alberts A S, Thorburn A M, Shenolikar S, Mumby M C and Feramisco J R. (1993). Proc. Natl. Acad. Sci. U.S.A., 90, 388-392.
Allard P, Zoubeidi A, Nguyen L T, Tessier S, Tanguay S, Chevrette M, Aprikian A and Chevalier S. (2000). Mol. Cell. Endocrinol., 159, 63-77.
Ayllon V, Cayla X, Garcia A, Fleischer A and Rebollo A. (2002). Eur. J. Immunol., 32, 1847-1855.
Ben-Dor I, Bern O, Tennenbaum T and Nir U. (1999). Cell Growth Differ., 10, 113-129.
Bennett D and Alphey L. (2002). Nat. Genet., 31, 419-423.
Berndt N, Dohadwala M and Liu C W. (1997). Curr. Biol., 7, 375-386.
Berridge, M. V., Herst, P. M., and Tan, A. S. (2005). Biotechnol. Annu. Rev. 11, 127-152]
Berthet C, Aleem E, Coppola V, Tessarollo L and Kaldis P. (2003). Curr. Biol., 13, 1775-1785.
Brugarolas J, Moberg K, Boyd S D, Taya Y, Jacks T and Lees J A. (1999). Proc. Natl. Acad. Sci. U.S.A., 96, 1002-1007.
Ceulemans H and Bollen M. (2004). Physiol. Rev., 84, 1-39.
Ceulemans H, Stalmans W and Bollen M. (2002). Bioessays, 24, 371-381.
Cheng M, Olivier P, Diehl J A, Fero M, Roussel M F, Roberts J M and Sherr C J. (1999). EMBO J., 18, 1571-1583.
Chow J P, Siu W Y, Fung T K, Chan W M, Lau A, Arooz T, Ng C P, Yamashita K and Poon R Y. (2003). Mol. Biol. Cell, 14, 3989-4002.
Connell-Crowley L, Harper J W and Goodrich D W. (1997). Mol. Biol. Cell, 8, 287-301.
Coqueret O. (2003). Trends Cell Biol., 13, 65-70.
Craig A W and Greer P A. (2002). Mol. Cell. Biol., 22, 6363-6374.
Craig A W, Zirngibl R, Williams K, Cole L A and Greer P A. (2001). Mol. Cell. Biol., 21, 603-613.
Dohadwala M, da Cruz e Silva E F, Hall F L, Williams R T, Carbonaro-Hall D A, Nairn A C, Greengard P and Berndt N. (1994). Proc. Natl. Acad. Sci. U.S.A., 91, 6408-6412.
Durfee T, Becherer K, Chen P L, Yeh S H, Yang Y, Kilburn A E, Lee W H and Elledge S J. (1993). Genes Dev., 7, 555-569.
Egloff M P, Johnson D F, Moorhead G, Cohen P T, Cohen P and Barford D. (1997). EMBO J., 16, 1876-1887.
Ezhevsky S A, Nagahara H, Vocero-Akbani A M, Gius D R, Wei M C and Dowdy S F. (1997). Proc. Natl. Acad. Sci. U.S.A., 94, 10699-10704.
Fischman K, Edman J C, Shackleford G M, Turner J A, Rutter W J and Nir U. (1990). Mol. Cell. Biol., 10, 146-153.
Garcia A, Cayla X, Guergnon J, Dessauge F, Hospital V, Rebollo M P, Fleischer A and Rebollo A. (2003). Biochimie, 85, 721-726.
Greer P. (2002). Nat. Rev. Mol. Cell. Biol., 3, 278-289.
Hao Q-L, Heisterkamp N and Groffen J. (1989). Mol. Cell. Biol., 9, 1587-1593.
Herrera R E, Sah V P, Williams B O, Makela T P, Weinberg R A and Jacks T. (1996). Mol. Cell. Biol., 16, 2402-2407.
Honkanen R E and Golden T. (2002). Curr. Med. Chem., 9, 2055-2075.
Iwanishi M, Czech M P and Chemiack A D. (2000). J. Biol. Chem., 275, 38995-39000.
Jirmanova L, Afanassieff M, Gobert-Gosse S, Markossian S and Savatier P. (2002). Oncogene, 21, 5515-5528.
Kaldis P. (1999). Cell Mol. Life Sci., 55, 284-296.
Katayama H, Zhou H, Li Q, Tatsuka M and Sen S. (2001). J. Biol. Chem., 276, 46219-46224.
Kim L and Wong T W. (1998). J. Biol. Chem., 273, 23542-23548.
Kitagawa M, Higashi H, Jung H K, Suzuki-Takahashi I, Ikeda M, Tamai K, Kato J, Segawa K, Yoshida E, Nishimura S and Taya Y. (1996). EMKO J., 15, 7060-7069.
Kogata N, Masuda M, Kamioka Y, Yamagishi A, Endo A, Okada M and Mochizuki N. (2003). Mol. Biol. Cell, 14, 3553-3564.
Kwon Y G, Lee S Y, Choi Y, Greengard P and Nairn A C. (1997). Proc. Natl. Acad. Sci. U.S.A., 94, 2168-2173.
Letwin K, Yee S-P and Pawson T. (1988). Oncogene, 3, 621-627.
Liu C W, Wang R H, Dohadwala M, Schonthal A H, Villa-Moruzzi E and Berndt N. (1999). J. Biol. Chem., 274, 29470-29475.
Ludlow J W, Glendening C L, Livingston D M and DeCarprio J A. (1993). Mol. Cell. Biol., 13, 367-372.
Lunter P C and Wiche G. (2002). Biochem. Biophys. Res. Commun., 296, 904-910.
Malumbres M, Sotillo R, Santamaria D, Galan J, Cerezo A, Ortega S, Dubus P and Barbacid M. (2004). Cell, 118, 493-504.
Miravet S, Piedra J, Castano J, Raurell I, Franci C, Dunach M and Garcia de Herreros A. (2003). Mol. Cell. Biol., 23, 7391-7402.
Morgenstern J P and Land H. (1990). Nucleic Acids Res., 18, 3587-3596.
Naviaux R K, Costanzi E, Haas M and Verma I M. (1996). J. Virol., 70, 5701-5705.
Orlovsky K, Ben-Dor I, Priel-Halachmi S, Malovany H and Nir U. (2000). Biochemistry, 39, 11084-11091.
Ortega S, Prieto I, Odajima J, Martin A, Dubus P, Sotillo R, Barbero J L, Malumbres M and Barbacid M. (2003). Nat. Genet., 35, 25-31.
Pan W, Sun T, Hoess R and Grafstrom R. (1998). Carcinogenesis, 19, 765-769.
Penhallow R C, Class K, Sonoda H, Bolen J B and Rowley R B. (1995). J. Biol. Chem., 270, 23362-23365.
Piedra J, Miravet S, Castano J, Palmer H G, Heisterkamp N, Garcia d H and Dunach M. (2003). Mol. Cell. Biol., 23, 2287-2297.
Priel-Halachmi S, Ben-Dor I, Shpungin S, Tennenbaum T, Molavani H, Bachrach M, Salzberg S and Nir U. (2000). J. Biol. Chem., 275, 28902-28910.
Reed S I. (1997). Checkpoint Controls and Cancer, Cancer Surveys 29. Kastern M (ed). Cold Spring Harbor Laboratory Press: Cold Spring Harbor, N.Y., pp. 7-23.
Rosato R, Veltmaat J M, Groffen J and Heisterkamp N. (1998). Mol. Cell. Biol., 18, 5762-5770.
Rubin E, Mittnacht S, Villa-Moruzzi E and Ludlow J W. (2001). Oncogene, 20, 3776-3785.
Salem Y, Shpungin S, Pasder O, Pomp O, Taler M, Malovani H and Nir U. (2005). Cell Signal., 17, 341-353.
Sellin J H, Umar S, Xiaq J and Morris A P. (2001). Cancer Res., 61, 2899-2906.
Sherr C J and Roberts J M. (2004). Genes Dev., 18, 2699-2711.
Shtutman M, Zhurinsky J, Simcha I, Albanese C, D'Amico M, Pestell R and Ben-Ze'ev A. (1999). Proc. Natl. Acad. Sci. U.S.A., 96, 5522-5527.
Sugiyama K, Sugiura K, Hara T, Sugimoto K, Shima H, Honda K, Furukawa K, Yamashita S and Urano T. (2002). Oncogene, 21, 3103-3111.
Taler M, Shpungin S, Salem Y, Malovani H, Pasder O and Nir U. (2003). Mol. Endocrinol., 17, 1580-1592.
Tetsu O and McCormick F. (2003). Cancer Cell, 3, 233-245.
Vermeulen K, Van Bockstaele D R and Berneman Z N. (2003). Cell Prolif., 36, 131-149.
Wang R H, Liu C W, Avramis V I and Berndt N. (2001). Oncogene, 20, 6111-6122.
Yamasaki L. (2003). Cancer Treat. Res., 115, 209-239.
Zarkowska T and Mittnacht S. (1997). J Biol. Chem., 272, 12738-12746.

Fer is an intracellular tyrosine kinase which was found to reside in both the cytoplasm and nucleus of mammalian cells (Ben-Dor et al., 1999; Hao et al., 1989; Letwin et al., 1988). Together with c-Fes, Fer constitutes a distinct subfamily of intracellular tyrosine kinases that share a unique structure. Both kinases bear an extended N-terminal tail which contains an Fps/Fes/Fer/CIP4 homology domain (FCH), followed by three coiled-coil-forming regions. While the FCH domain could mediate the association of Fer and c-Fes with microtubular structures, the coiled-coil domains were shown to direct the oligomerization of these kinases. The kinase domain of the two enzymes resides at their carboxy terminal part and is 70% identical in the two proteins (Greer, 2002).

Fer is activated by growth factors such as EGF and PDGF in fibroblastic cells (Kim and Wong, 1998), and by occupation of the Fey receptor in mast cells (Penhallow et al., 1995). Activation of Fer in these systems could be linked to the modulation of cell-cell and cell-substratum interactions, since Fer was shown to associate with and phosphorylate, adherence molecules (Craig et al., 2001; Greer, 2002; Kim and Wong, 1998; Kogata et al., 2003; Miravet et al., 2003; Piedra et al., 2003; Rosato et al., 1998). Fer was also shown to associate with key cellular regulatory proteins like phosphatidylinositol-3 kinase (PI3K) (Iwanishi et al., 2000), signal transducer and activator of transcription-3 (Stat3) to (Priel-Halachmi et al., 2000), and with the cytoskeletal linker protein plectin (Lunter and Wiche, 2002). In the case of PI3K, Fer binds the active form of this enzyme in insulin-treated adipocytes (Iwanishi et al., 2000), corroborating the involvement of Fer in insulin signaling pathways (Taler et al., 2003).

Although present in a wide variety of tissues and cells, the functional role of Fer has been elucidated mainly in cells which carry out innate immune responses (Craig and Greer, 2002; Greer, 2002). Mice devoid of an active Fer develop normally and the proliferation of fibroblasts derived from these mice is not impaired in vitro (Craig et al., 2001).

Fer was detected in all human malignant cell lines analyzed (Hao et al., 1989; Orlovsky et al., 2000) and its levels in malignant prostate tumors are significantly higher then those detected in benign prostate tumors (Allard et al., 2000). Furthermore, down-regulation of Fer impaired the proliferation and abolished the ability of prostate carcinoma PC3 cells to form colonies in soft agar (Allard et al., 2000). U.S. patent application Ser. No. 10/486,101 having Publication Number 20050063973 discloses short interfering RNA (siRNA) molecules directed to sequences of the fer gene capable. These siRNA molecules were found to inhibit the growth of PC3 cells and to arrest tumor growth in an animal model.

### SUMMARY OF THE INVENTION

In one of its aspects, the present invention provides a method and system for altering or inhibiting the effects of the Fer protein in a cell, and particularly in a malignant cell. This aspect of the invention is based upon the novel and unexpected finding that Fer can associate with the PP1α isoform of protein phosphatase 1 (PP1). The inventors have found that association of Fer with PP1α leads to phosphorylation of the PP1α which inhibits the phosphatase activity of the PP1α. As a result of the inhibition of the PP1α activity, the pool of phosphorylated retinoblastoma protein (pRB) increases resulting in cell proliferation and cancer. In accordance with this aspect of the invention, the effect of the Fer protein in cells is altered or modulated by any one or more of the following:

(a) modulating or altering the association of Fer to PP1;

(b) modulating or altering the phosphorylation of PP1;

(c) modulating or altering the phosphatase activity of PP1; and

(e) modulating or altering the tumor suppressive activity of the retinoblastoma protein (RB).

In another of its aspects, the invention provides a method and system for assessing or monitoring the effect of the Fer protein in a cell. In accordance with this aspect of the invention, the effect of the Fer protein in a cell is assessed or monitored by assessing or monitoring the level of any one or more of the following:

(a) the association of Fer to PP1;

(b) the phosphorylation of PP1;

(c) the phosphatase activity of PP1; and

(e) the tumor suppressive activity of the retinoblastoma protein (RB).

In yet another of its aspects, the present invention proves a system for detecting substances capable of modulating, altering or inhibiting the one or more effects of Fer in malignant cells. This aspect of the invention is based upon the novel and unexpected finding that yeast cells expressing the fer cDNA from mouse show a significant decrease in growth rate in comparison to yeast cells expressing the plasmid alone. In accordance with this aspect of the invention, determining whether a substance is capable of modulating, altering or inhibiting the effects of Fer comprises presenting the substance to cells expressing an exogenous fer expressing yeast cells. A significant change in the growth rate of the fer expressing yeast cells indicates that the substance is capable of modulating, altering or inhibiting the effects of Fer and is thus capable of altering a Fer mediated pathway. The substance may be a low molecular weight compound. In a preferred embodiment of this aspect of the invention, a plurality of substances are screened for their ability to alter a Fer mediated pathway in 96 well plates, wherein each well yeast cells expressing the fer cDNA are inoculated together with a chemical substance as a potential inhibitor. The wells are subsequently screened to locate those wells in which the Fer expressing cells have a growth rate similar to that of the controls.

In another of its aspects, the invention provides a method for inhibiting the growth in a mammalian cell expressing a fer gene, comprising;
(a) Altering or inhibiting the function of a Fer protein in the cell.
(b) Inhibiting the association of a Fer protein to PP1 in the cell;
(c) Inhibiting the phosphorylation of PP1 in the cell;
(d) Activating the phosphatase activity of PP1; and
(e) Activating the tumor suppressive activity of the retinoblastoma protein (RB) in the cell.

The invention also provides a method for treating cancer in an individual comprising altering or inhibiting one or more effects of a Fer protein.

In yet another of its aspects, the invention provides a method for determining whether a substance is capable of altering or inhibiting an effect of a Fer protein in a cell, comprising:
(a) presenting the substance to a first cell expressing an exogenous fer gene or fer cDNA; and
(b) measuring a rate of proliferation of the cell; and
(c) comparing the rate of proliferation of the cell to a rate of proliferation of a second cell not expressing an exogenous fer gene or fer cDNA,
wherein a significant difference between the growth rate of the first cell and the growth rate of the second cell indicating that the substance is capable of altering a fer mediated pathway.

The invention also provides a cell expressing an exogenous fer gene or a fer cDNA.

The invention also provides a pharmaceutical composition for the treatment of cancer comprising a compound having a molecular weight of up to 1000 Dalton capable of inhibiting an effect of a Fer protein and a physiologically acceptable carrier.

### BRIEF DESCRIPTION OF THE DRAWINGS

In order to understand the invention and to see how it may be carried out in practice, embodiments will now be described, by way of non-limiting example only, with reference to the accompanying drawings, in which

FIG. 1 shows Western blot analysis of whole cell lysates (WCL) from PC3 cells that were either left untreated (lane 1) or exposed for 72 h to luc. (lane 2), or fer-siRNA (lane 3), reacted with anti-Fer or anti-Actin antibodies;

FIG. 2 shows flow-cytometry analysis of PC3 cells untreated or exposed to luc. or fer-siRNA;

FIG. 3 shows Western blot analysis of CDK2 levels in Fer depleted prostate carcinoma cells;

FIG. 4 shows relative levels of CDK2 RNA in total RNA from untreated, luc.-siRNA treated and from fer-siRNA treated cells determined using quantitative RT-PCR analysis

FIG. 5 shows SDS-PAGE of WCL prepared from untreated, luc. or fer-siRNA-treated PC3 cells;

FIG. 6 shows hypo-phosphorylation of PP1α in fer-siRNA treated PC3 cells (A) and in fer-siRNA treated MDA-MB-231 cells (B);

FIG. 7 shows Consensus PP1α binding motifs in the kinase domains of the human Fer, the murine Fer and FerT proteins (A), and immunoprecipition of Myc-PP1α from lysates of MDA-MB-231 cells (B);

FIG. 8 shows western blot analysis of whole cell lysates of MCF-7 cells over expressing HA-fer or infected with the pBabe vector alone;

FIG. 9 shows comparison of growth rate of SP1 yeast cells expressing the pAES426 HA-fer plasmid with yeast cells expression the blank plasmid;

FIG. 10 shows expression of HA-fer in yeasts;

FIG. 11 shows Western blot analysis in Yeast using an antiphosphotyrosine antibody;

FIG. 12 shows comparison of growth rate in SP1 Yeast cells expressing the pAES426 HA-Fer plasmid, the pAES426 HA-fer Y715F plasmid and the empty expression rector;

FIG. 13 shows expression of HA-fer and HA-fer Y715F in Yeast by Western blot analysis using an anti HA-epitope antibody;

FIG. 14 shows phospho-tyrosine levels in yeasts by Western blot analysis using an anti phosphotyrosine antibody;

FIG. 15 shows results of screening 30 substances for their ability to interfere with Fer function in yeast;

FIG. 16 shows retesting of seven substances that tested positively in the yeast assay;

FIG. 17 shows viability of PC3 prostate carcinoma cells untreated (left bar), subjected to DMSO alone (middle bar), and grown in the presence of the compound 218B7 (right bar);

FIG. 18 shows cell growth in DU145 prostate carcinoma cells, untreated cells (left bar), cells exposed to DMSO alone (middle bar), and cells grown in the presence of the compound 218B7 (right bar);

FIG. 19 shows cell growth in the breast cancer cell line MDA-MB-231, untreated cells (left bar), cells exposed to DMSO alone (middle bar), and cells grown in the presence of the compound 218B7 (right bar); and

FIG. 20 shows cell growth in the breast cancer cell line MCF-7, untreated cells (left bar), cells exposed to DMSO alone (middle bar), and cells grown in the presence of the compound 218B7 (right bar).

### DETAILED DESCRIPTION OF EMBODIMENTS

### Materials and Methods

Preparation of siRNA

Double-stranded RNAs, 21 nucleotides long, were synthesized by Dharmacon Research (Lafayette, Colo., USA). The targeted sequence was derived from the human fer cDNA (accession no. J03358) 5′ AAA GAA ATT TAT GGC CCT GAG 3′ (nt 84-104 relative to the fer mRNA translation initiation codon). Custom SMART pool siRNA to target the human cdk2 mRNA and the human cdk4 mRNA were purchased from Dharmacon. Selected siRNAs sequences were submitted to a BLAST search against the human genome sequence to ensure specificity of the siRNA. A sequence targeting firefly (Photinus pyralis) luciferase (luc.) gene (Accession no. X65324) was used as a control.

Cell Lines and Transfection of Cells

Human PC3 and MDA-MB-231 cells were grown at 37° C. in RPMI and MCF-7 cells were grown in DMEM (Gibco-Invitrogen). All growth media were supplemented with 10% FCS.

For siRNA transfection, 1.5×10⁵ PC3 cells were seeded in 6 cm Petri dish one day before transfection. 30 μl siRNA from 20 μM stock solution were mixed with 300 μl OptiMEM (Gibco-Invitrogen) medium, then incubated at room temperature for 5 min. 20 μl Metafectene (Biontex) were mixed with 160 μl OptiMEM in a separate tube and were also incubated at room temperature for 5 min. The two mixtures were then combined, gently mixed, and incubated for another 15 min at room temperature, for the formation of lipids-siRNA complexes. Growth medium was removed from the cells, which were then covered with 1290 μOptiMEM, 200 μl FCS, and 510 μl lipid-siRNA mixture. Final siRNA concentration in the Petri dish was 300 nM. 8 hours later, 1800 μl Optimem and 200 μM FCS were added and cells were harvested 72 h after transfection.

For siRNA transfection, 1.4×10⁵ MDA-MB-231 cells were seeded in 6 cm Petri dish one day before transfection. OptiMEM (300 μl) was mixed with 30 μl of 20 μM siRNA duplex and with 7 μl of PolyMAG (Magnetofection-Chemicell GmBH) and the mixture was left for 5 min at room temperature. After addition of 1663 μl Opti-MEM, the entire mixture was added to the cells and the culture dish was laid on a manufacturer supplied magnet plate (MagnetoFACTORE plate) for an additional 15 min at 37° C. The transfection mixture was then removed and the transfected cells were grown in RPMI supplied with 10% FCS, for 72 h.

For transient over-expression of PP1α, 7×10⁵ MDA-MB-231 or 1×10⁶ MCF-7 cells were seeded in 10 cm plates one day before transfection. Each transfection was performed by mixing 12 μg DNA with 12 μl PolyMAG in 200 μl Opti-MEM medium and incubated for 5 min at room temperature. After addition of 5 ml Opti-MEM medium, the entire mixture was added to the cells and the culture dish was put on a magnet plate for additional 15 min at 37° C. The transfection mixture was then removed and the transfected cells were grown in RPMI supplemented with 10% FCS. Cells were harvested for protein extraction 48 h after beginning of transfection.

For transient over-expression of HA-Fer, HA-FerF606A/F649A, or pcDNA3 expression vector alone, PC3 cells were seeded in 10 cm plates one day before transfection. Each transfection was performed by mixing 15 μg DNA in 1 ml Opti-MEM medium and mixtures were left for 5 min at room temperature. 30 μl Lipofectamine 2000 (Invitrogen) were mixed with 1 ml OptiMEM in a separate tube and were also incubated at room temperature for 5 min. The two mixtures were then combined, gently mixed, and incubated for another 30 min at room temperature. After addition of 8 ml Opti-MEM medium, the entire mixture was added to the cells for 6 h at 37° C. The transfection mixture was then removed and the transfected cells were grown in RPMI supplemented with 10% FCS. Cells were harvested for protein extraction 48 h after beginning of transfection.

MCF-7 cells over-expressing hemaglutinin-tagged Fer (HA-Fer) were established as follows: 293T cells were co-transfected with 10 μg pHAFer (Salem et al., 2005) and 10 μg of the pCL-Ampho packaging plasmid (Naviaux et al., 1996). Production of viruses was performed as described before (Morgenstern and Land, 1990). For retroviral infection, 2×10⁵ MCF-7 cells were seeded in 6 cm plates and were then infected with undiluted 293T supernatants, in the presence of 8 μg/ml polybrene (Sigma) for 6 h. This process was repeated three times in succession, producing an MOI (multiplicity of infection). Two days later, cells were split and selected in puromycin up to a concentration of 2 μg/ml. Cells transfected with the pBabe puro vector alone were used as a control.

For treatment with cycloheximide, PC3 cells transfected with siRNA for 72 h, were subjected to 10 μg/ml cycloheximide for additional 4 h, at the end of the transfection process.

Quantitation of Viable Cells.

Relative number of viable cells in a given culture was determined using the MTT assay. Cultures were assayed for the production of formazan by the reduction of MTT (3-(4,5-dimethylthiazol-2-yl)-2,5-diphenyl tetrazolium bromide). 72 h after transfection in 96-well plates, growth medium was removed and cells were incubated for 2 h with 100 μl MTT solution (5 mg/ml MTT dissolved in PBS) at 37° C. Cells were then lysed with 50% N,N-dimethyl formamide and the levels of the formazan reduction product were determined by measuring absorbance at 570 nm in a computer-controlled microplate analyzer.

Flow Cytometry Analysis

Cells were rinsed twice with cold phosphate buffer saline (PBS), and then harvested by treatment with trypsin. Suspended cells were washed twice in 10 ml cold PBS and collected at 500×g for 5 min. The pellet was resuspended in 200 μl cold PBS and cells were stained in the dark with 200 μl propidium iodide (PI) solution (20 mM Tris, pH 8, 1 mM NaCl, 0.1% (v/v) NP-40, 1.4 mg/ml RNase A, 0.05 mg/ml propidium iodide) for 30 min at 37° C. The total cellular DNA content was determined using a Becton Dickinson flow cytometer (FACSCalibur), Multiple software and ModFit LT software.

BrdU Incorporation Assay

PC3 cells were grown in 6 cm Petri dish and 72 h after transfection with RNAi, were incubated for 30 min with 10 μM BrdU (Boehringer Mannheim). Cells were then washed twice with cold PBS, fixed for 30 min with cold ethanol (70%) in ice. Fixed cells were incubated for 30 min in 2 M HCl/0.5% Triton-X100. The cells were centrifuged and resuspended in 0.1 M Na₂B₄O₇*10H₂O, pH 8.5. The cells were spun down and resuspended in blocking solution (1% bovine serum albumin in PBS/0.5% Tween) for 30 min at room temperature in the presence of 1:50 diluted anti-BrdU monoclonal antibody (347580, Becton Dickinson). Cells were centrifuged again, then incubated under the same conditions with 1:100 fluorescein isothiocyanate (FITC)-conjugated goat anti-mouse antibody (Jackson ImmunoResearch Labs). The cells were centrifuged and stained in the dark with 5 μg/ml PI solution. The cellular DNA content was determined using a Becton Dickinson flow cytometer (FACSCalibur) and Multiple software.

Immunoprecipitation

Protein lysates (700 μg) were incubated overnight at 4° C. with 1:100 diluted anti-c-Myc (13-2500, Zymed), or anti-PP1 antibodies (sc-443 and sc-7482, Santa Cruz). Antigen-antibody complexes were precipitated with protein A-Sepharose (Amersham Biosciences) after 2-h incubation at 4° C. Precipitates were washed 3 times with TGET buffer (20 mM Tris HCl, 10% glycerol, pH 7.5 EDTA 1 mM, 0.1% Triton X-100). The first wash was carried out with TGET buffer supplemented with 150 mM NaCl, the second wash was done with TGET containing 75 mM NaCl, and the last wash was with TGET devoid of NaCl.

Western Blot Analysis

Whole cell lysates (WCL) were prepared as described before (Salem et al., 2005). For Western blot analysis, 20-30 μg proteins from each sample were resolved in 7-12% SDS-PAGE. Electroblotted proteins were detected using specific anti-Fer antibodies (Priel-Halachmi et al., 2000), anti-CDK2 (sc-6248, Santa Cruz), anti-CDK1 (sc-54, Santa Cruz), anti-CDK4 (610148, BD Biosciences), anti-CDK6 (554086, BD Biosciences), anti-CDK7 (sc-856, Santa Cruz), anti-cyclin A (sc-751, Santa Cruz), anti-cyclin D1 (sc-6281, Santa Cruz), anti-cyclin E (sc-247, Santa Cruz), anti-cyclin H (sc-1662, Santa Cruz), anti-pRB (sc-102, Santa Cruz), anti-phosphosthreonine⁸²¹-pRB (pThr821) (44-582, Biosource), anti-phosphoserine⁷⁹⁵-pRB (pSer795) (sc-7986-R, Santa Cruz), anti-phosphoserine⁷⁸⁰-pRB (pSer780) (9307, Cell Signaling), anti-phosphoserine^{807/811}-pRB (pSer807/811) (9308, Cell Signaling), anti-PP1α (2582, Cell Signaling), anti-PP1 (sc-443, Santa Cruz), anti-phosphothreonine³²⁰-PP1α (pThr320) (2581, Cell Signaling), anti-p21Cip1 (sc-6246, Santa Cruz), anti-p27Kip1 (sc-528, Santa Cruz), anti-HA (NMS-101R, Covance), anti-Myc (13-2500, Zymed), and anti-Actin antibody (A 4700, Sigma). Bound antibodies were visualized using chemiluminescence reaction (Pierce). The blots were scanned using an optical scanner—Umax Astra 3400 and the optical density of each band was compiled using the ImageJ software application.

Quantitative RT-PCR Analysis

Whole cell RNA was extracted from PC3 cells using TRI Reagent (Molecular Research Center, ICN) following the manufacturer's instructions. 25 ng of total RNA was reverse-transcribed using AccessQuick RT-PCR System (Promega) with specific oligonucleotides derived from the human CDK2 mRNA: forward primer 5′ CTA GCT TTC TGC CAT TCT CAT C 3′ and reverse primer 5′ GAA GAG CTG GTC AAT CTC AGA A 3′. The selected primers are derived from separated exons (4 and 6), which flank an intron of the CKD2 gene. PCR was run for 27 cycles which were found to be optimal for quantitative comparison of the CDK2 mRNA levels. The expected 275 by long PCR product is specific to the mature CDK2 mRNA, and can be differentiated from a contamination of genomic DNA PCR products. 18S ribosomal RNA primer pairs (Quantum RNA, Ambion) were used to yield a 489 by fragment derived from the 18S rRNA (ribosomal RNA) as an internal control. PCR products were separated in 1.4% agarose gel and visualized with ethidium bromide.

Construction of Plasmids

The pCMV-c-Myc-PP1α expression plasmid was constructed by amplifying a human PP1α cDNA clone (RZDP—IRAUp969F0224D) with a forward primer 5′ AACCCGAATTCTGTCCGACAGCGAGAAGC 3′, which bears at it's 5′ end a EcoR1 site, and a backward primer-5′ AAGGACTCGAGCTATTTCTTGGCTTTGGCGG 3′, which bears a Xho1 site at is 3′ end. The amplified fragment was cloned into a Ecor1-Xho1 cut pCMV-c-Myc vector (BD Clonetech Inc.).

Mutated Fer (FerF606A/F649A) was constructed by ‘site directed mutagenesis’ method as follows. In the first step, Phe⁶⁰⁶ was changed to Ala as follows: primers A and B are reverse compliment primers that contain the mutated residue: A—5′ AAAATAAAGGCTCTACAGGAAGC 3′ and B—5′ GCTTCCTGTAGAGCCTTTATTT 3′. Primers C and D are the external 5′ and 3′ primers designed for the amplification of the mutated cDNA portions: C—5′ GCATGACTGGTACCATGGTG 3′ and 5′ TTCTTGCTCTCGAGATACAACA 3′. The two overlapping cDNA portions were separately amplified from a murine fer-cDNA and were than mixed, denatured and allowed to complete a mutated double-stranded fragment using Pfu Ultra DNA polymerase. The obtained mutated fer fragment was then amplified and cloned into a corresponding fer-cDNA to give rise to a mutated FerF606A. In the next step, Phe⁶⁴⁹ was replaced with Ala as follows: primers E and F are reverse compliment primers that contain the mutated residue: E—5′ TTTCTGACCGCCCTGAGG 3′ and F 5′ CCTCAGGGCGGTCAGAAA 3′. The same external primers C and D were used as described above. The two overlapping cDNA portions were separately amplified from the FerF606A cDNA and were than mixed, denatured and allowed to complete a mutated double-stranded fragment using Pfu Ultra DNA polymerase. The obtained mutated fragment was then amplified and cloned into a corresponding Fer F606A cDNA to give rise to a double mutated Fer (FerF606A/F649A).

In Vitro Phosphatase Assay

MCF-7 cells over-expressing HA-Fer or harboring the retroviral vector pBabe as control were transiently transfected with a Myc-PP1α expression vector. WCL were prepared from the cells 48 h after transfection in a lysis buffer which lacked phosphatase inhibitors. Myc-PP1α was immunoprecipitated from 1 mg lysate with anti-c-Myc antibody as described above. The immunoprecipitated Myc-PP1α was rinsed twice with buffer A (50 mM Tris HCl, 5% glycerol, 50 mM NaCl, 1 mM DTT, 1 mM MgCl₂, 3 mM MnCl₂). The immune complexes (containing each 150 ng protein) were then suspended in a phosphatase activity buffer (buffer A containing 0.1 mg/ml purified BSA) and were then incubated at 30° C. in the presence of the synthetic substrate 5 mM pNPP disodium hexahydrate (Sigma). Dephosphorylation of the substrate was quantitated by measuring absorbance at 405 nm in a computer-controlled microplate analyzer.

Yeast Strain and Growth Conditions

Experiments were carried out using baker's yeast, Saccharomyces cerevisiae SP1 strain having the following genotype: MATa, trp1, ade8, can1, his3, leu2, ura3. (Cameron et al., 1988). Growth of the yeast prior to transformation was done on ypd medium. After transformation, growth was done on selective ynb media lacking uracil (as described in Current Protocols in Molecular Biology).

Plasmid and Cloning:

The plasmid pAES 426 served as the expression vector in yeast and is a derivative of the plasmid pADNS (Colicelli et al, 1998), and it contains the constituative promoter ADH (alcohol dehydrogenase), 80H terminator, μ2 ori MCS (Multiple Cloning Sites), a gene conferring resistance to ampicillin, to allow selection with bacteria, and JN, a gene encoding for ura3, to allow selection in yeast.

PECE HA-Fer plasmid which expresses the mouse HA-Fer, conjugated at its N terminus to the HA epitope under the control of the SV40 early gene promoter, serves to remove the fer cDNA to allow introduction into the expression vector of the yeast. PECE HA-Fer Y715F plasmid expressing mouse Fer, in which the tyrosine amino acid 715 has been replaced with the amino acid phenylalanine. This Fer mutant is unable to autophosphoralate and is therefore inactive (Ben-Dor et al., 1999) conjugated at its N terminus to the HA epitope under the control of the SV 40 early gene promoter. This plasmid served to remove the mutant region in the HA-Fer Y715F cDNA, in order to allow introduction of the expression vector in yeast.

Construction of the Plasmid pAES 426 HA-Fer:

The fer cDNA was removed from the plasmid PECE HA-Fer using the restriction enzyme SPEI (BCU1) and Bgl2 and was purified on 1% agarose gel using a kit obtained from Promega. The target plasmid pAES 426 was also cut by the same two restriction enzymes and after cutting CLAP (calf intestine alkaline phosphatase) was added in order to remove the phosphate group from the 5′ end of the plasmid. Removal of the phosphate groups prevents the plasmid from closing on itself, unless an insert has been introduced during the ligation step. The ligation was carried out at a molecular ratio of 1:3 between the vector and the insert, respectively, Following ligation, using a ligase from Fermentas Inc. (5 U/μg DNA), the mixture underwent a transformation to competent E. coli bacteria, using heatshock for 90 seconds at a temperature of 42° C. The bacterial cells were then inoculated on LB plates containing empicillin at a concentration of 100 μl/ml. In order to test the product, several colonies were selected which were grown on liquid LB medium containing ampicillin at a concentration of 100 microliters per millimeter overnight at 37° C. with mild shaking. From these bacteria, the plasmid was produced using the mini-prep kit of Bioneer, Inc. Extracted plasmids were cut with XhoI in order to verify the presence of the insert in the pAES 426 vector. The constructed plasmid was termed pAES 426 HA-Fer.

Construction of the Plasmid pAES 426 HA-Fer Y715F:

In order to construct this plasmid vector, a segment of the fer cDNA was removed from the PECE HA-Fer plasmid and was replaced with a fragment removed from the plasmid PECE HA-Fer Y715F, containing the point of mutation in the amino acid at position 715. For this purpose, use was made of the restriction enzyme Sad for cutting the two plasmids. The remainder of the process was carried out similarly to that described above for the construction of the plasmid pAES 426 HA-Fer.

Transformation to Yeast

This was done using the lithium acetate method, as described in Molecular Genetics of Yeast. In general, the yeast was grown to an O.D. of about 1 (600 nanometers) and was centrifuged and resuspended in 50 microliters of lithium acetate −100 mM.

The yeast were incubated at 30° C. for 45 minutes with mild shaking with: 45 microliters of the DNA intended for the transformation, 240 microliters PEG 50%, 36 microliters 1 M lithium acetate, and 25 microliters fish sperm that served as a DNA carrier. The yeast cells subsequently underwent heat shock at 42° C. for 25 minutes. At the end of the process, the yeast were centrifuged and resuspended in 100 microliters of sterile water and inoculated on selection plates.

Extraction of Protein from Yeast:

Yeast cells were grown to an OD of about 1 (600 nM) centrifuged and resuspended in 10 ml of 20% TCA (Trichloro acetate). After an additional centrifugation, the yeast were resuspended in 200 microliters 20% TCA. An equal volume of glass beads was added to the mixture which was then vortexed for 8 minutes in order to disrupt the cell wall of the yeast. The liquid obtained was transferred to a new Eppendorf test tube, and the beads were washed two additional times with 5% TCA in a volume of 200 microliter. The liquid was added to the Ependorf test tube, so that the final concentration of the TCA was 10%. The contents of the Eppendorf test tube were centrifuged and the supernatant was discarded. The pellet was resuspended in 75-100 microliter Laemeli sample buffer X2, and after that 37.5 to 50 microliters Tris base 1 molar PH 8.6 was added. After mixing, the samples were boiled for 3 minutes, centrifuged and the supernatant was collected.

Determination of Protein Concentration:

5-20 microliters of protein samples were obtained as described above, applied to a comb prepared by cutting Whatman 3 mm paper, 10 cm in length with “teeth” about 0.5 cm wide and a space of about 0.1 cm between each teeth. In parallel, duplicates of the protein BSA (Bovine Serum Albumin) from a 2 ml per liter stock solution were loaded on a separate comb in increasing amounts. The combs were introduced to Coomassie staining solution for 30 minutes with mild shaking. At the end of the staining, the combs were destained until a light background was obtained around the samples. The combs were dried on an aluminum foil, placed on a heating block. Each tooth containing a sample of yeast protein extract was cut and placed separately in 0.5 milliliters of 3% SDS in wells of a 24 well plate. The plate was placed for shaking for one hour at 37° C. or overnight at room temperature. From each well 200 microliters was transferred to a 96 well plate and the color intensities were read in an ELISA reader at a wavelength of 590 nanometers. A calibration curve was prepared using the wells containing the BSA and the amount of protein was calculated in the samples accordingly.

Western Blot Analysis:

Samples of 15-30 microgram protein were separated in 10% SDS-PAGE. Low range protein markers (Biorad Inc.) were run in parallel. At the end of the separation process, the proteins were transferred by an electrical field to a nitro cellulose membrane, using transfer buffer and a fixed voltage of 15 volts for 20 to 25 minutes. The separation and the transfer were carried out in a Semi-Dry system of Bio-Rad. The efficiency of the transfer to the membrane was checked using a Ponceu staining. In order to detect specific proteins via the binding of specific antibodies, a blocking buffer was used containing 3-5% skimmed milk (in accordance with the type of antibody used) and 0.1% Tween 20 in PBS (Phosphate Buffere saline). Membranes were left in this buffer for 1 hour at room temperature. After blocking, an incubation was carried out with a primary antibody diluted in blocking buffer for 1 hour at room temperature or overnight at 4° C. At the end of the incubation, three rinses were carried out in PBS-T (PBS containing 0.1% Tween 20), each one for 5 minutes. At the end of the rinses, a second antibody was added, conjugated to peroxidase, obtained from Jackson laboratories. Membranes were incubated with the secondary antibody diluted in blocking buffer for 1 hour at room temperature. At the end of the incubation, three rinses were carried out in PBS-T each for 5 minutes. Visualisation of the proteins that reacted with the antibody specifically, was done using a chemoluminescence amplification system (ECL) from Pierce Inc. in accordance with the manufacturer's instructions.

Screening System:

HTS (high throughput screening). Yeasts expressing the murine Fer were inoculated in 250 microliters of liquid growth medium in wells of 96 well plates. The concentration of the yeasts that were inoculated was 0.0005 OD. To each well, 5 miroliters of a particular chemical substance diluted in DMSO from a compound library (Chemical Diversity Ltd.) at a final concentration of 10 micromolar, were added. The yeasts were grown at 37° and the growth rate was followed by measuring OD at 600 nanometers as a function of time including the readings at time 0. In addition, in each plate the following controls were carried out:

Yeasts expressing the mouse Fer gene together with 5 microliters DMSO.

Yeasts expressing the empty vector without fer cDNA, and 5 microliters DMSO.

Growth medium YNB-ura without yeasts.

Yeasts expressing the Fer Y715 F mutant in the presence of 5 microliters DMSO.

Results

Down-Regulation of Fer Arrests Malignant Cells at the G0/G1 Phase

To dissect the molecular role of Fer, the cellular level of this kinase was specifically reduced in two malignant cell lines which harbor a functional retinoblastoma protein (pRB): PC3: a prostate carcinoma cell-line, and MDA-MB-231: a breast carcinoma cell-line. The two cell-lines were exposed for 72 h to a synthetic double-stranded small interfering RNA (siRNA) directed towards a unique 21 nt sequence in the human fer mRNA (Hao et al., 1989). In parallel, the cells were treated with a non-relevant siRNA directed toward the luciferase (luc.) mRNA.

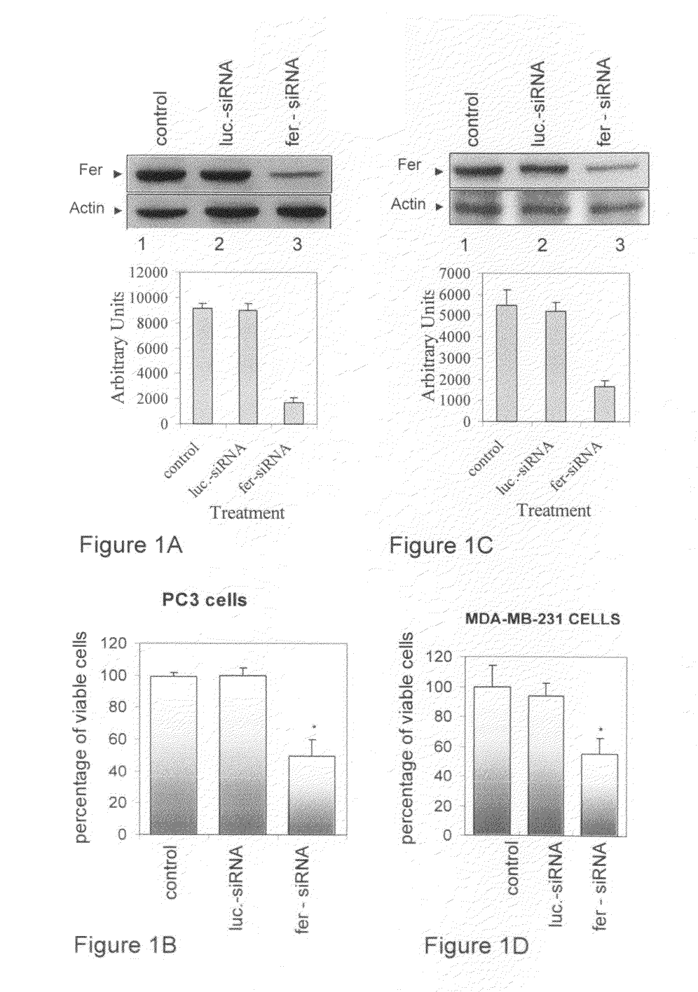

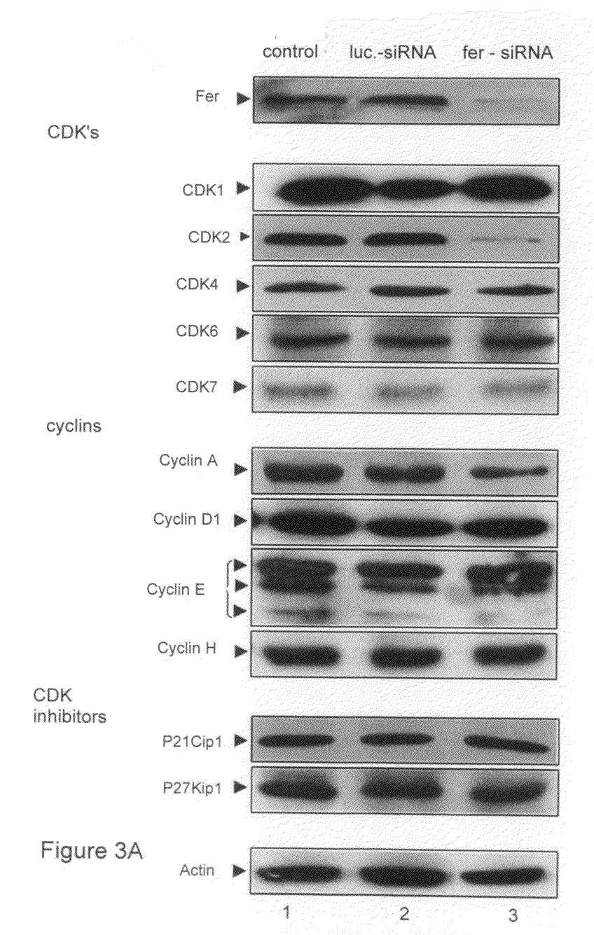

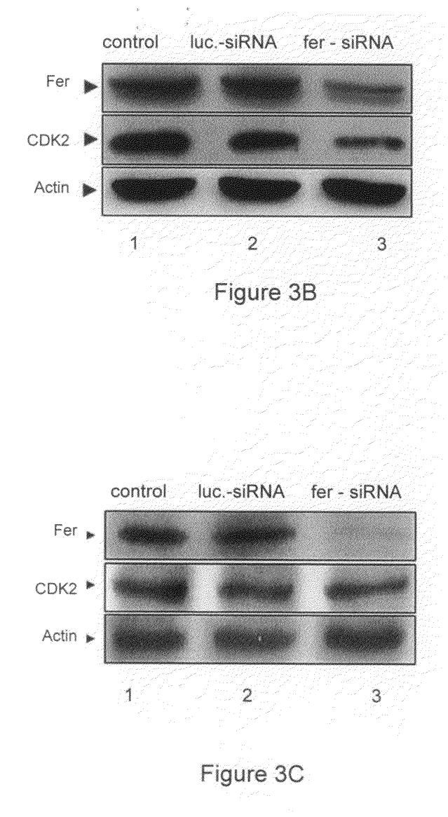

FIG. 1A shows a western blot analysis in which whole cell lysates (WCL) from PC3 cells were either left untreated (lane 1) or exposed for 72 h to luc. (lane 2), or fer-siRNA (lane 3) and then reacted with anti-Fer or anti-Actin antibodies. Scanning values of radiograms from three independent experiments are presented as mean±S.E. (*P<0.001 compared to luc.-siRNA, calculated using a Student's t-test). FIG. 1B shows an MTT viability assay of cultures of PC3 cells that were untreated, or exposed to luc.-siRNA, or fer-siRNA. The values represent percentage of viable cells compared to the control and are means±S.E. [*p<0.01 (N=5) compared to luc.-siRNA treated cells]. In FIG. 1C, WCL from MDA-MB-231 that were either untreated, or treated with luc., or fer-siRNA were reacted with anti-Fer and anti-Actin antibodies in a western blot analysis. Scanning values of radiograms from three independent experiments are presented as means±S.E. (*P<0.005 compared to luc.-siRNA). MDA-MB-231 cells, untreated or exposed to luc.-siRNA, or fer-siRNA were subjected to a MTT viability assay. Values represent means±S.E. [*p<0.005 (N=5) compared to luc.-siRNA treated cells]. The results shown in FIG. 3 demonstrate that exposure of either cell line to the fer-siRNA led to a specific decrease of about 5- and 3-fold in the level of Fer in PC3 and MDA-MB-231 cells, respectively. fer-siRNA did not affect the level of actin which was used as a control (FIGS. 1A, C). A decrease in the level of fer lowered the number of cells in PC3 and MDA-MB-231 treated cultures by approximately 50 and 40% respectively, as was determined using the 3-(4,5-dimethylthiazol-2-yl)-2,5-diphenyl tetrazolium bromide (MTT) assay (FIGS. 1B, D). This could either result from a diminished viability of the treated cells or could reflect their impaired proliferation rate.

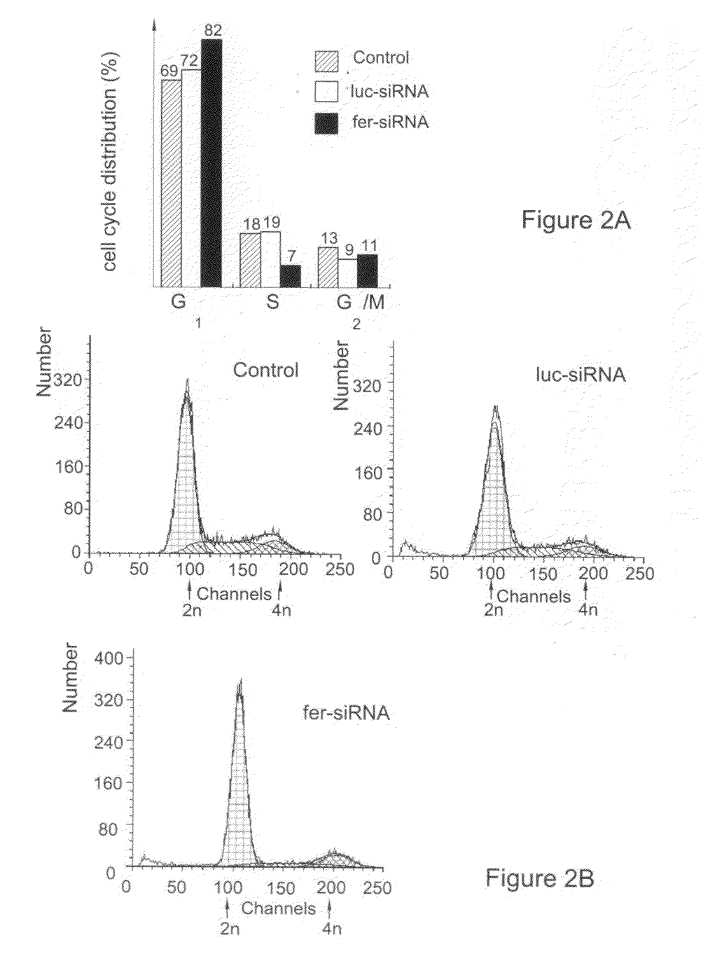

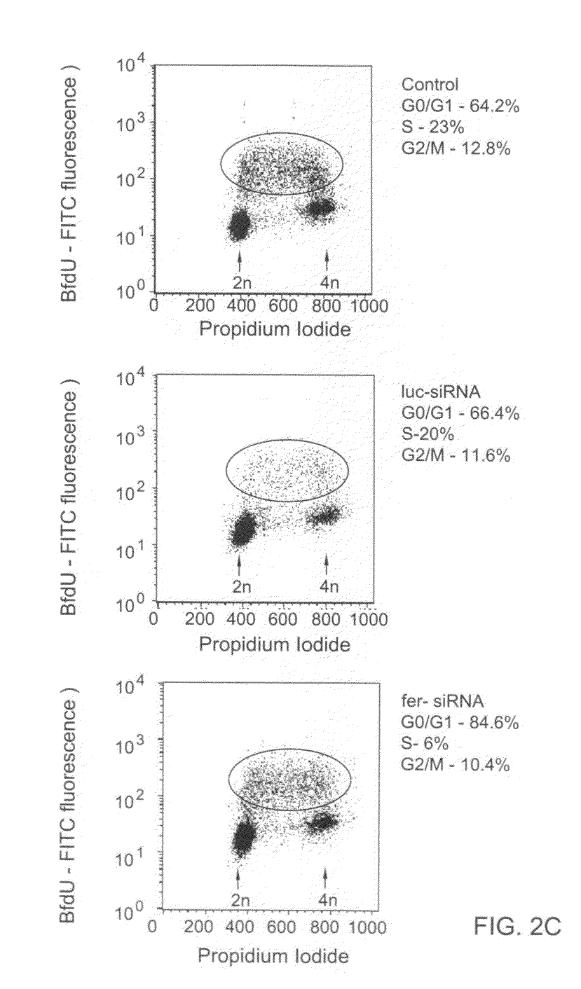

A flow cytometry analysis was carried out on PC3 cells that were either untreated or exposed to luc. or fer-siRNA. FIG. 2A shows a histogram of the percentage of cells in each cell-cycle phase. The flow-cytometry distribution profiles of the values presented in the histogram are shown in FIG. 2B. The peaks of 2n and 4n DNA containing cells are indicated by arrows. In FIG. 2C, control and treated cells were incubated with BrdU and the percentage of cells incorporating BrdU (ellipses) was determined. The values represent one out of three independent experiments that gave similar results.

The flow-cytometry analysis shown in FIG. 2 demonstrates that while luc.-siRNA did not affect the cell-cycle profile of PC3 cells, fer-siRNA increased the percentage of the G0/G1 cells and markedly decreased the percentage of cells to residing in the S phase from 19 to 7% (FIG. 2A). However, neither luc. nor fer-siRNA significantly affected the percentage of the sub-G0/G1 apoptotic cells in the treated cultures (FIG. 2B). Moreover, annexin V and LDH (lactate dehydrogenase) assays corroborated the lack of apoptotic or necrotic death induction, in the fer-siRNA treated cells (data not shown). This implies that depletion of fer affects the transition of cells from the G1 to the S phase rather than affecting their viability.

To confirm the essential role of fer in the G1—S transition, untreated and siRNA-treated PC3 cells were subjected to Bromo-deoxy-Uridine (BrdU) incorporation assay. While the percentage of S phase cells was similar in the untreated and in luc.-siRNA treated cells, fer-siRNA treatment reduced the percentage of BrdU incorporating cells from 20 to 6% (FIG. 2C). Hence, reduction in the cellular level of Fer interferes with the transition of PC3 cells from the G0/G1 to the S phase. A similar reduction in S phase population was also observed in fer-siRNA treated MDA-MB-231 cells (data not shown).

Knock-Down of Fer Leads to the Down-Regulation of CDK2 in Prostate Carcinoma Cells

The arrest of Fer-deprived cells in the G0/G1 phase suggests the involvement of Fer in maintaining or modulating the function of G1-S regulatory proteins. The cellular levels of both negative and positive regulators of G1 progression and G1-S transition (Vermeulen et al., 2003) were therefore determined in untreated and in siRNA-treated cells. PC3 cells were subjected to the fer-siRNA and the levels of early G1 cyclins like cyclin D1, the late G1 cyclin—cyclin E and the S phase cyclin—cyclin A (Vermeulen et al., 2003), were determined in fer-siRNA and in luc.-siRNA treated cells.

FIG. 3 shows the effect of luc.-siRNA or fer-siRNA on cultures of PC3 cells. In panel A, the cells were left untreated, and the levels of the indicated proteins were determined in a western blot analysis. In panel B, DU145 cells were left untreated or treated with luc.-siRNA or fer-siRNA and levels of the indicated proteins were determined using western blot analysis. These profiles represent one out of three independent experiments that gave similar results. FIG. 3C shows that the level of CDK2 is not changed in fer-siRNA treated MDA-MB-231 cells. These profiles represent one out of three independent experiments that gave similar results.

Of the three cyclins analyzed, only the level of cyclin A was slightly decreased in fer-siRNA treated cells (FIG. 3A). We also examined the levels of CDKs that serve as a catalytic constituent of the cyclin/CDK complexes (Vermeulen et al., 2003). While the levels of CDK4, CDK6, and CDK1 were not changed in cells subjected to the fer-siRNA, the level of CDK2 was significantly reduced in PC3 and in another prostate carcinoma cell line DU145 which was subjected to fer-siRNA (FIG. 3A, B). Interestingly, the level of CDK2 was not changed in fer-siRNA treated MDA-MB-231 cells (FIG. 3C). In parallel, the levels of the CDK-activating kinase (CAK) components—CDK7 and cyclin H were also not affected (FIG. 3A), suggesting a lack of an effect on the functional activation of CDKs (Kaldis, 1999) upon “knock-down” of Fer. In accordance with this conclusion we did not see any reduction in the in vitro kinase activity of CDK4 or CDK6 in Fer depleted cells (data not shown). The levels of the negative regulators of CDKs—p21 Cip1 and p27Kip1 (Coqueret, 2003), were also not altered by the down-regulation of Fer (FIG. 3A).

To determine whether Fer regulates the accumulation of CDK2 at the transcriptional or post-transcriptional level, reverse transcribed-polymerase chain reaction (RT-PCR) analysis was carried out and the levels of the CDK2 mRNA were determined in luc.-siRNA and in fer-siRNA treated cells.

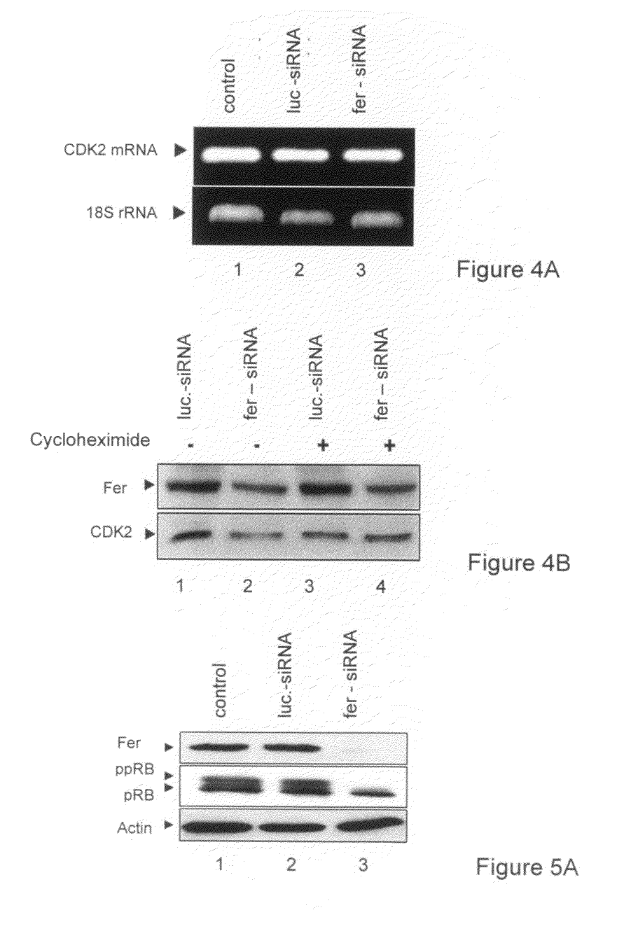

The relative levels of CDK2 RNA in total RNA from untreated, luc.-siRNA treated and from fer-siRNA treated cells were determined using quantitative RT-PCR analysis. The results are shown in FIG. 4A. 18S ribosomal RNA served as an internal control. In FIG. 4B, PC3 cells were treated with luc.-siRNA or with fer-siRNA in the absence or presence of cycloheximide. Levels of Fer and CDK2 in whole cell lysates were determined using western blot analysis. These data represent one out of three independent experiments that gave similar results. The results shown in FIG. 4 show that there is not a significant difference between the levels of the CDK2 mRNA in the two analyzed samples (FIG. 4A). Furthermore, simultaneous exposure of PC3 cells to both fer-siRNA and the translation inhibitor cycloheximide did not exacerbate the effect of fer-siRNA on the level of CDK2, but rather alleviated this effect (FIG. 4B lanes 2 and 4). These findings suggest that CDK2 is regulated by Fer at the post-translational level in prostate carcinoma cells.

pRB is Hypo Phosphorylated in Fer-SiRNA-Treated Cells

CDK2 associates with cyclin E and cyclin A to constitute kinase active complexes during G1 and S phase progression (Brugarolas et al., 1999). These two complexes, together with CDK4-6/cyclinD1 (Ezhevsky et al., 1997) are engaged in the onset and maintenance of the phosphorylation state of pRB. Phosphorylation of pRB releases E2Fs (Reed, 1997; Yamasaki, 2003) which in turn activate transcription of genes involved in the onset and regulation of DNA replication (Herrera et al., 1996). The phosphorylation state of pRB in untreated, luc.-siRNA and in fer-siRNA-treated PC3 and MDA-MB-231 cells was therefore checked.

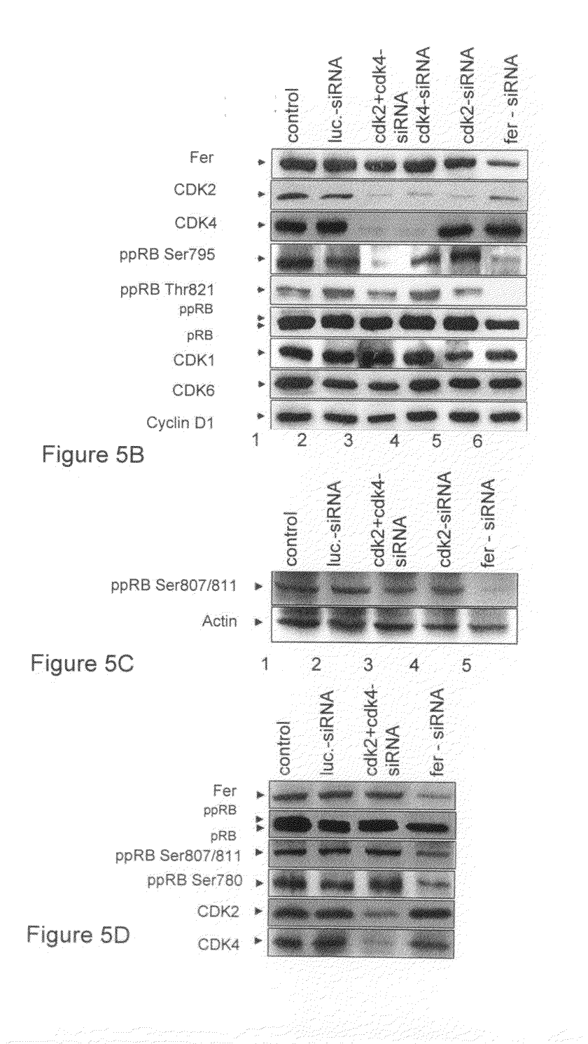

In FIG. 5A, WCL prepared from untreated, luc. or fer-siRNA-treated PC3 cells were resolved in a 7-17% gradient SDS-PAGE. Levels of Fer, pRB, ppRB (phosphorylated RB) and Actin were determined. In FIGS. 5B. and C, PC3 were left untreated, or were treated with a luc.-siRNA, with a combination of cdk2 and cdk4 siRNAs, with a cdk4-siRNA, with a cdk2-siRNA, or with a fer-siRNA. Levels of the indicated proteins or pRB. phosphorylation sites were determined using western blot analysis. The data represent one out of three independent experiments that gave similar results. In FIG. 5D, whole cell lysates were prepared from untreated, luc.-siRNA treated, cdk2 and cdk4-siRNA treated, or fer-siRNA treated MDA-MB-231 cells. Levels of indicated proteins and pRB phosphorylation sites were determined in a western blot analysis. The data represent one out of three independent experiments that gave similar results.

While in untreated and in luc.-siRNA treated cells, unphosphorylated and a pool of phosphorylated pRB forms were clearly detected (FIG. 5A), in fer-siRNA treated cells the phosphorylated fraction of pRB (ppRB) was dramatically reduced (FIG. 5A). The nearly complete de-phosphorylation_ of pRB in Fer depleted PC3 cells, indicated that both CDK2 and CDK4/6 sites are de-phosphorylated in the treated cells. To directly examine this possibility the phosphorylation levels of several sites, were determined. Ser795 is a key regulatory site in pRB (Connell-Crowley et al., 1997) and it is preferentially phosphorylated by CDK4 (Pan et al., 1998). Thr821 on the other hand is a preferential CDK2 phosphorylation target (Zarkowska and Mittnacht, 1997). The phosphorylation levels of these two sites were determined using specific anti-phospho antibodies. While the phosphorylation level of these two sites was only slightly changed upon depletion of either CDK4 or CDK2 alone (FIG. 5B), both sites were de-phosphorylated in the fer-siRNA treated cells (FIG. 5B). Moreover, simultaneous depletion of CDK4 and CDK2 affected only the phosphorylation level of Ser795 but not the phosphorylation state of Thr821 (FIG. 5B). Similarly, knock-down of Fer significantly decreased the phosphorylation level of two other CDK2 phosphorylation sites Ser807 and Ser811 (Connell-Crowley et al., 1997), whose phosphorylation was shown to be necessary for G1-S transition (Brugarolas et al., 1999) (FIG. 5C). To substantiate the pivotal role of Fer in maintaining the phosphorylation state of pRB in malignant cells, similar analysis was conducted in MDA-MB-231 cells. The phosphorylation levels of Ser780 which is a primary phosphorylation site of CDK4 (Kitagawa et al., 1996), and of Ser807/811 were examined. While knock-down of Fer significantly affected the phosphorylation states of these CDK4 and CDK2 phosphorylation targets, simultaneous depletion of CDK4 and CDK2 hardly affected the phosphorylation levels of these sites (FIG. 5D). Interestingly, the phosphorylation levels of Thr821 and Ser795 were not affected by the knock-down of Fer or CDK4 and CDK2 in MDA-MB-231 cells (data not shown), indicating the variations in pRB regulation in different cell types. Hence, down-regulation of Fer leads to the hypo-phosphorylation of pRB.

Knock-Down of Fer Leads to the Hypophosphorylation of PP1α

Maintaining the phosphorylation state of pRB, independently of CDK6, CDK4 and CDK2, implied the involvement of Fer in an alternative pRB regulating pathway. While G1/S CDKs phosphorylate pRB on defined sites (Connell-Crowley et al., 1997; Kitagawa et al., 1996; Zarkowska and Mittnacht, 1997), the protein phosphatase 1 (PP1) isoforms de-phosphorylate these sites in pRB (Rubin et al., 2001). The balance between these two opposing regulatory mechanisms determines the phosphorylation and activation state of pRB (Berndt et al., 1997). It was therefore examined whether Fer controls the activation state of the PP1α isoforms (Wang et al., 2001) in vivo. Phosphorylation of Thr320 in PP1α suppresses the phosphatase activity of that enzyme and increases the phosphorylation level of Thr320 which is inversely correlated with the phosphatase activity of PP1α in vivo (Dohadwala et al., 1994; Kwon et al., 1997; Liu et al., 1999). Conversely, de-phosphorylation of the Thr320 site induces the phosphatase activity of PP1α toward pRB (Berndt et al., 1997). The phosphorylation state of PP1α on Thr320 in Fer depleted cells was therefore examined.

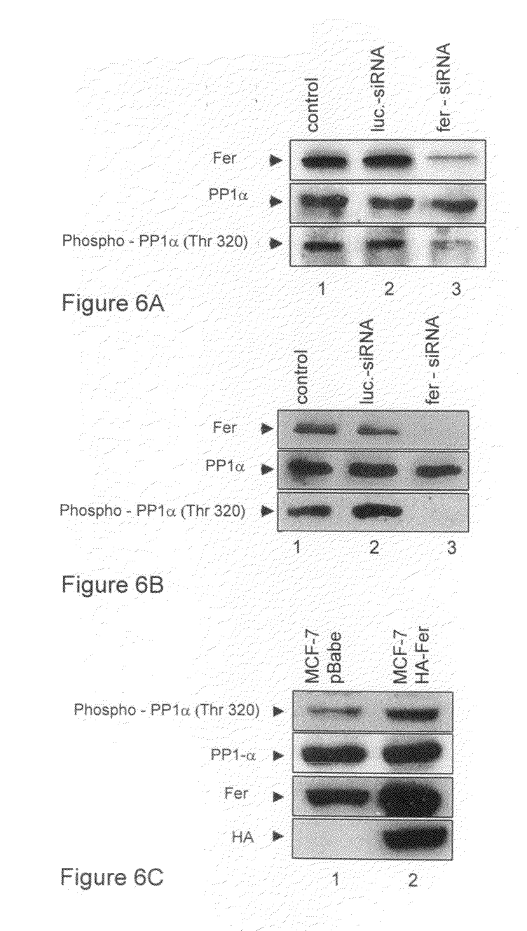

WCL of PC3 cells (FIG. 6A) or MDA-MB-231 cells (FIG. 6B) were subjected to anti-Fer, anti-PP1α, or anti-pThr320 PP1α in a western blot analysis. WCL were reacted with anti-Fer, anti-PP1α, or anti-pThr320 PP1α. In FIG. 6C, whole cell lysates from MCF-7 cells infected with the pBabe vector and from MCF-7 cells ectopically expressing HA-Fer were reacted with anti-pThr320 PP1α, anti-PP1α, anti-Fer, and anti-HA antibodies, in a western blot analysis. The data represent one out of three independent experiments that gave similar results. The results shows that knockdown of fer in PC3 cells did not change the cellular level of PP1α but led to a profound decrease in the phosphorylation level of PP1α on Thr320 (FIG. 6A). Similarly, knockdown of Fer decreased the phosphorylation level of PP1α in MDA-MB-231 cells (FIG. 6B).

To corroborate the regulatory link between Fer and PP1α a reciprocal experiment was carried out. HA-tagged Fer (HA-Fer) was stably over-expressed in MCF-7 breast carcinoma cells and the effect of this ectopic expression on the PP1. Thr320 phosphorylation level, was examined. Over-expression of Fer up-regulated the phosphorylation of PP1α on Thr320 (FIG. 6C) but did not affect the cellular level of PP1α. This implies a regulatory link between Fer and PP1α.

PP1α Associates with Fer and is Down-Regulated in Fer Over-Expressing Cells

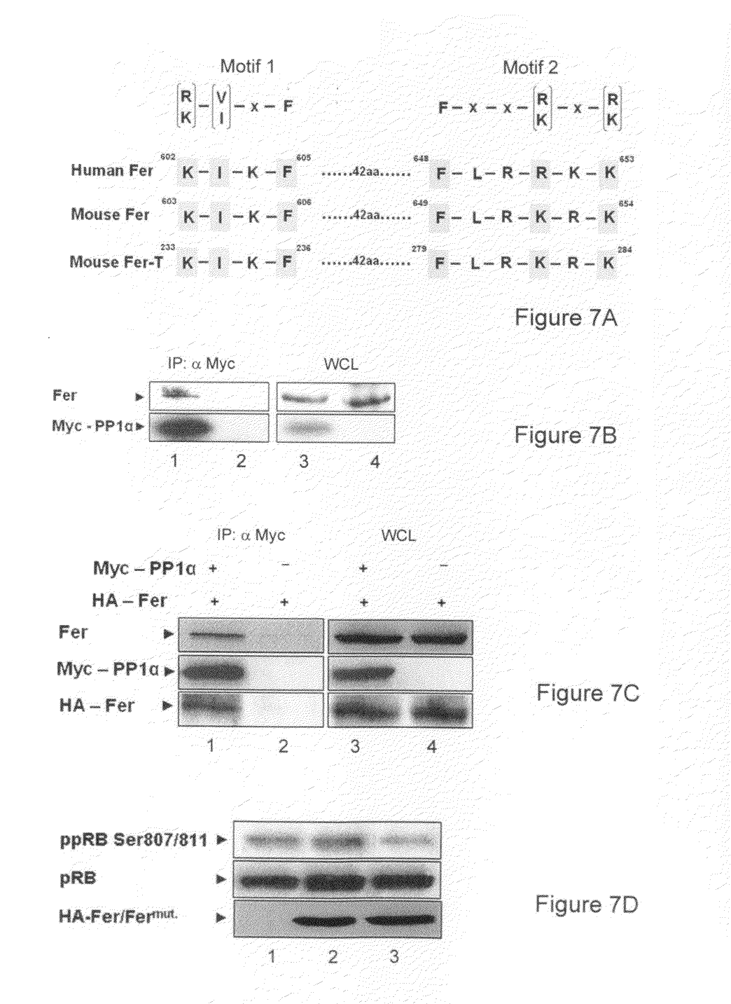

The effect of Fer depletion on the activation state of PP1α suggested a direct interaction between Fer and PP1. The PP1 consensus binding site has been thoroughly characterized in a plethora of proteins and was accurately defined (Egloff et al., 1997). The presence of two PP1 docking motifs was identified in various PP1 binding proteins (Ayllon et al., 2002; Bennett and Alphey, 2002; Garcia et al., 2003; Sugiyama et al., 2002). Careful analysis of the murine and human Fer amino acid sequence revealed the presence of the two PP1 docking motifs in the kinase domain of the two kinases. FIG. 7A shows consensus PP1α binding motifs in the kinase domains of the human Fer, the murine Fer and FerT proteins, where the numbers denote the position of each motif in the Fer proteins. As shown in FIG. 7A, in the two proteins the PP1 binding motifs are separated by 42 amino acids from each other. As expected, the two PP1 binding motifs are present also in the kinase domain of the meiotic testes specific Fer variant-FerT (FIG. 7A) (Fischman et al., 1990).

To examine whether Fer and PP1 can indeed associate with each other in vivo, MDA-MB-231 cells were transiently transfected with a vector encoding a Myc-tagged PP1α catalytic subunit or with the pCMV expression vector alone and the association of the Myc-PP1α protein with the endogenous fer was determined using co-immunoprecipitation analysis. The cells were transfected with a Myc-PP1α (lanes 1 and 3 in FIG. 7B) or with the expression vector alone (lanes 2 and 4). Myc-PP1α was immunoprecipitated from cell lysates using anti-Myc antibodies. Precipitated proteins (lanes 1 and 2) and WCL (lanes 3 and 4) were reacted with anti-Fer antibodies, in a western blot analysis. The membranes were then re-probed with anti-Myc antibody.

While Fer co-precipitated with Myc-PP1α from cells expressing the ectopic phosphatase, it was not precipitated by the anti-Myc antibodies from lysates of cells transfected with the expression vector alone (FIG. 7B). To corroborate this finding MCF-7 cells stably over-expressing HA-Fer were transiently transfected with the Myc-PP1α encoding vector and Myc-PP1α was immunoprecipitated from cells lysates using anti-Myc antibodies. MCF-7 cells over-expressing HA-Fer were transfected with a Myc-PP1α plasmid (lanes 1 and 3 in FIG. 7C) or with the expression vector alone (lanes 2 and 4). Myc-PP1α was immunoprecipitated from cell lysates using anti-Myc antibodies. Precipitated proteins (lanes 1 and 2) and WCL (lanes 3 and 4) were reacted with anti-Fer antibodies (upper panels), in a western blot analysis. Membranes were then re-probed with anti-Myc and anti-HA antibody.

As was found in MDA-MB-231 cells, Fer co-precipitated with the Myc-PP1α protein (FIG. 7C), showing the association of Fer with PP1α in vivo. To further establish the link between Fer-PP1 interaction and the pRB phosphorylation state, PC3 cells were transiently transfected with a vector expressing a native HA-Fer, and with a vector encoding a mutated murine Fer in which a highly conserved phenylalanine residue in each PP1 binding motif (Katayama et al., 2001) was replaced with alanine (HA-FerF606A/F649A). This double mutation compromised the ability of Fer to bind PP1.

Referring to FIG. 7D, PC3 cells were transiently transfected with either a pcDNA3 expression vector alone (lane 1), or with a vector encoding a native HA-Fer (lane 2), or the HA-FerF606A/F649A mutant (lane 3). Whole cell lysates were prepared from all samples and were reacted with anti-phosphoserine^{807/811}-pRB (upper panel), anti-pRB (middle panel) and anti-HA antibodies, in a western blot analysis. This data represents one out of three independent experiments that gave similar results.

Ectopic expression of the Fer mutant led to a decrease in the phosphorylation of pRB on serines 807/811 (FIG. 7D). This decrease was only seen following the expression of the HA-FerF606A/F649A mutant but not with the native enzyme (FIG. 7D, lane 2) or with a kinase inactive Fer mutant—FerY715F which lacks an autophosphorylation and substrate phosphorylation activity (Ben-Dor et al., 1999; Salem et al., 2005), but retains the ability to bind PP1 (data not shown). These results show a link between the ability of Fer to bind PP1 and the phosphorylation state of the cell-cycle regulator pRB, in malignant cells.

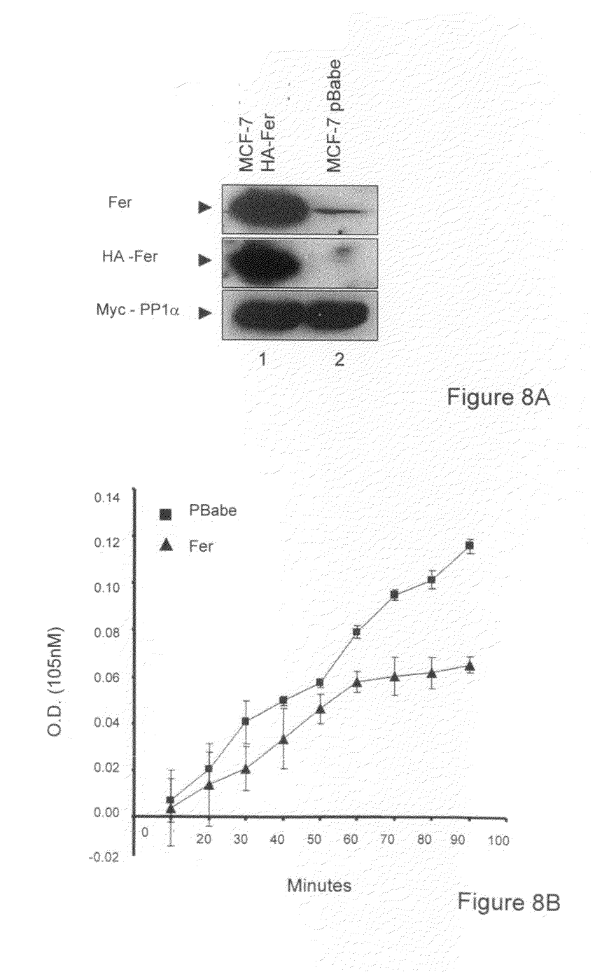

To directly examine the effect of Fer on the phosphatase activity of PP1α, Myc-PP1α was transiently expressed in MCF-7 cells which ectopically express HA-Fer and in MCF-7 cells that harbor the expression vector pBabe alone. The cells were transiently transfected with a Myc-PP1α expression plasmid. Whole cell lysates were reacted with anti-Fer, anti-HA, and anti-Myc antibody, in a western blot analysis. (FIG. 8A). These two cell populations accumulate different levels of Fer (FIG. 8A) but exhibit similar patterns of cell-cycle progression (data not shown). Myc-PP1α was immunoprecipitated from the lysates in FIG. 8A. using anti-Myc antibody. The precipitates were subjected to phosphatase in vitro assay for the indicated periods of time. In FIG. 8B, squares represent MCF-7-pBabe cells and triangles denote MCF-7-HA-Fer cells. Values represent means±S.E. [P<0.05 (n=3) when HA-Fer is compared to pBabe after 90 min incubation in 30° C.]. Myc-PP1α was immunoprecipitated from cell lysates and was then subjected to an in vitro phosphatase assay. Incubation of the precipitated enzymes for 90 min in the presence of a synthetic phosphatase substrate revealed a two fold decrease in the phosphatase activity of PP1α that was precipitated from MCF-7-HA-Fer extracts (FIG. 8B). Thus, over-expression of Fer decreases the phosphatase activity of PP1α.

Screening System in Yeast

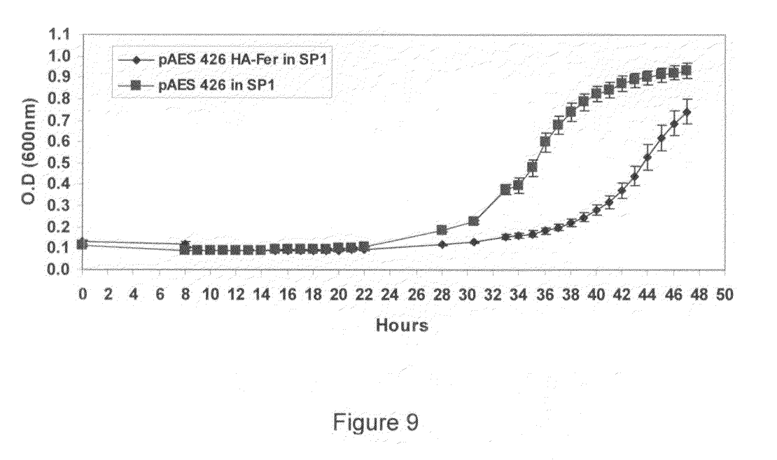

In order to test the effect of the expression of murine Fer in yeast cells, the plasmid PAES 426 HA-Fer was introduced into the wild-type strain of Saccharomyces cerevisiae SP1. The growth rate of the yeast cells was examined in liquid medium on 96 well plates, and reading OD with a wavelength of 600 nanometers. FIG. 9 shows that differences in growth rate were observed between the yeast expressing the Fer and the controls starting from 22 hours after inoculation, at a level of significance of P<0.001 (N=3). These differences increased until the yeast entered the stationary phase. After transformation, an identical number of colonies was observed in comparison to the number of colonies observed on control plates into which the blank vector was introduced without the cDNA of the mouse. However, the colonies expressing the fer proliferated at a slower rate and the size of the colonies was smaller in comparison to the colonies of the yeast cells expressing the blank vector.

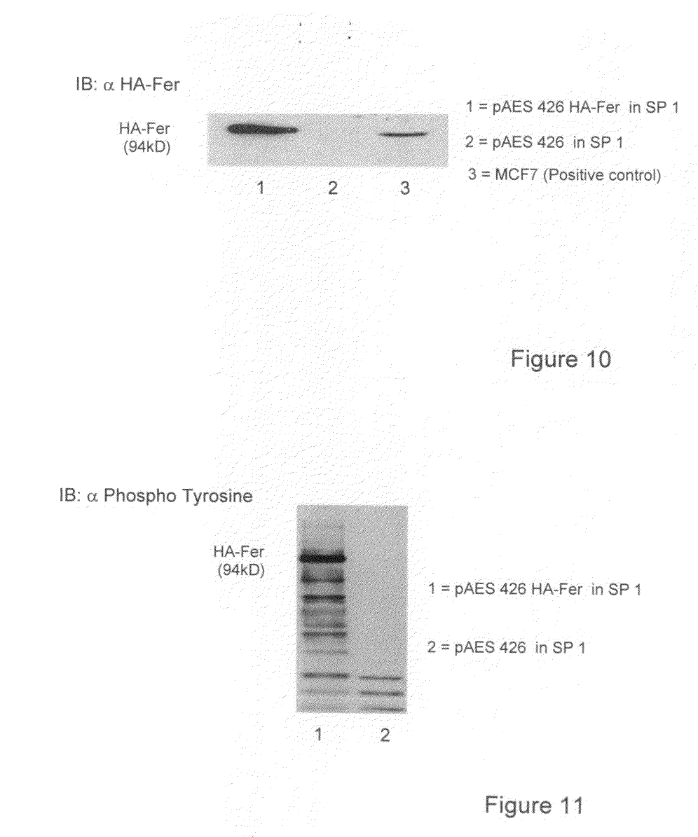

In order to verify the presence of the fer, expression levels of the kinase were examined by Western Blot using an antibody against the HA epitope, fused to the N terminus of the Fer that was introduced into the yeast cells. Western blot analysis with anti HA epitope antibody. Referring to FIG. 10, protein extracts were prepared from: Yeasts expressing HA-Fer (lane 1), yeasts containing an empty expression vector (lane 2), and proteins from MCF-7 human breast carcinoma cells (column 3) Which served as a positive control.

In order to examine the phosphorylation state of Fer and to examine whether its presence influences the phosphorylation level of yeast cell proteins, a Western Blot analysis was done using the α phosphotyrosine antibody. As can be seen in FIG. 11, exogenous Fer was autophosphorylated and is thus active. Nonetheless, there is the possibility that the labeled band is of a yeast protein having the same migration rate on gel, Similarly, phosphorylation of yeast proteins were observed that apparently are due to the forced expression of Fer.

Inhibition of the growth of yeast, expressing the vector pAES 429 HA-Fer, in comparison to yeast expressing the blank vector, is apparently the result of the forced presence of the Fer that is even active in the yeast. Apparently, the fer kinase phosphorylates tyrosine residues in endogenous yeast proteins involved in proliferation, and may thus bring about an inhibition of proliferation.

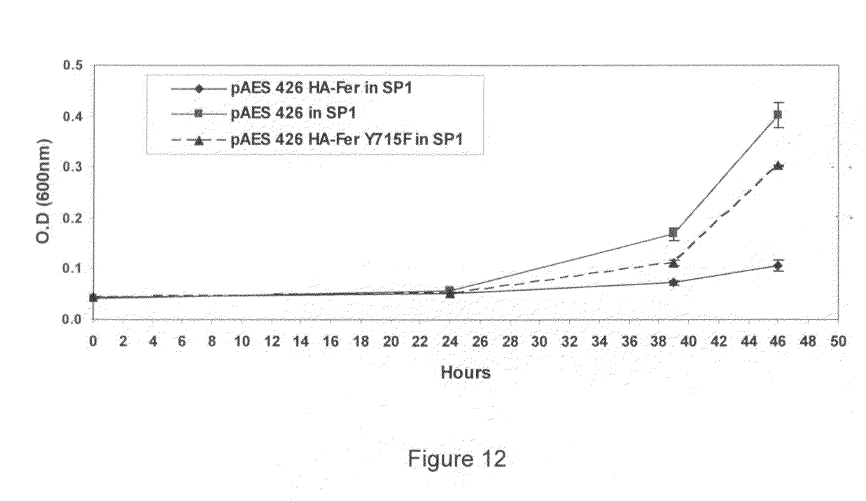

Expression of inactive Fer (HA-Fer Y715 F) does not cause inhibition of growth in yeast. In order to examine whether the activity of the Fer kinase is the cause of the inhibition of cell growth, the growth rate of the yeast expressing the HA-Fer Y715 F was compared to the growth rate of yeast expressing the HA-Fer and yeast expressing the MT plasmid (FIG. 12). The graph shows the average of three independent experiments which gave similar results. Differences in growth rate starting from 39 hours post inoculation are significant (p<0.0005) for yeast expressing the pAES426 HA-Fer Y715F plasmid in comparison to yeasts containing the empty expression vector.

The growth rate of yeast expressing the protein HA-Fer Y715 F was practically identical to the control and a significant inhibition of growth rate was not observed. In contrast to this, 39 hours after inoculation, a difference was observed at a level of significance of p≦0.005 (M=3), in the growth rate of the yeast expressing the protein HA-Fer in comparison to yeast expressing the protein HA-Fer Y715 F. As can be seen, 46 hours after inoculation, the differences between yeast expressing the active kinase in comparison to those expressing the inactive kinase, increased significantly.

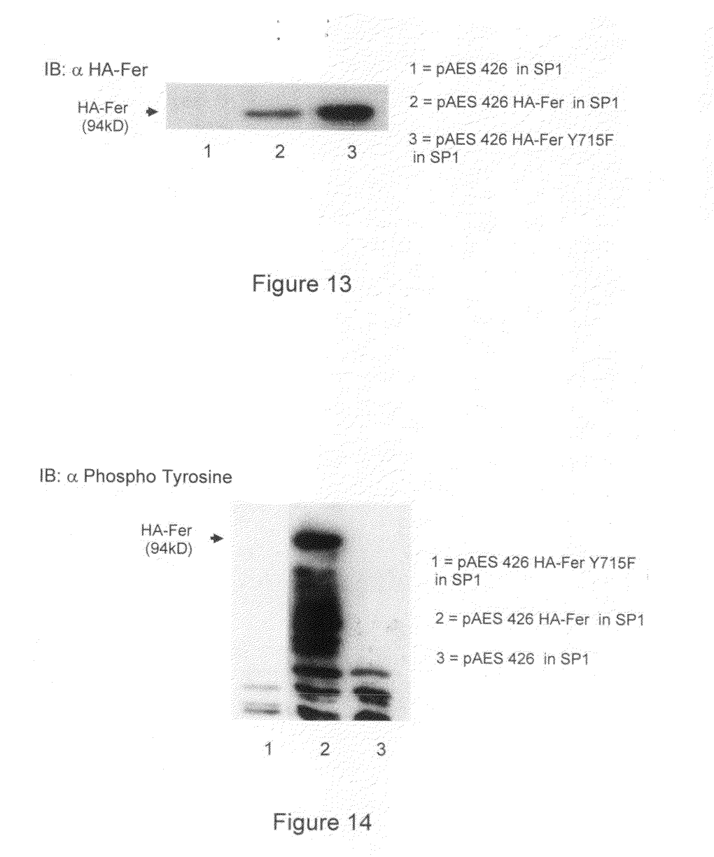

At the molecular level, the expression levels of the proteins were examined by using an antibody against the HA epitope (FIG. 13), and the extent of phosphorylation of the tyrosine residue by use of the anti phosphotyrosine antibody (FIG. 14). FIG. 13 shows the expression of the Fer kinase and of the mutant kinase in the active site, in yeast SP1 cells. Protein extracts were prepared from: yeasts expressing HA-Fer Y715F (lane 1), yeasts expressing HA-Fer (lane 2) and yeasts harboring an empty expression vector (lane 3).

FIG. 14 shows that yeast proteins are not phosphorylated in yeast expressing the HA-Fer Y715 F and this is in contrast to yeast expressing the active fer kinase.

These results show that the imposed presence of an active exogenous Fer in yeast leads to a phenotype of inhibited growth involving expression of this enzyme. The presence of this protein in its inactive form does not affect the growth rate in yeast and this inhibition of growth rate is evidently a result of the phosphorylation on tyrosine residues, carried out by Fer on yeast proteins.

Screening for Inhibitors of Fer

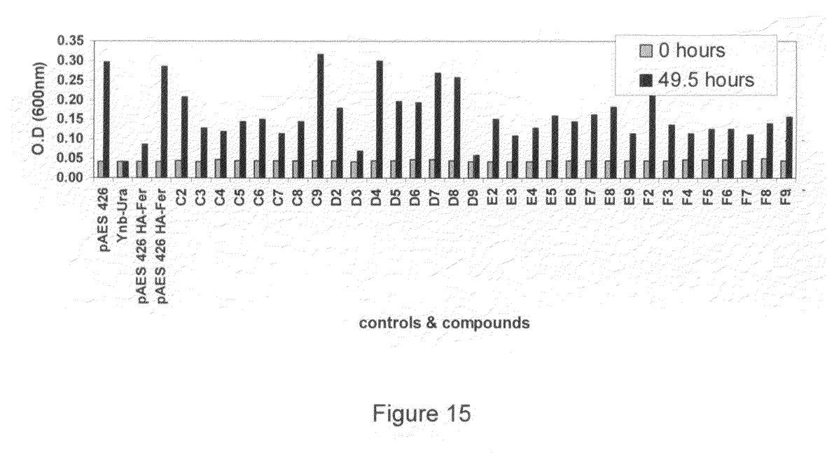

A sublibrary consisting of 3,000 substances having a molecular weight of up to 1000 Dalton from a library of 50,000 substances was obtained from Chemical Diversity, San Diego, Calif. The substances were screened for their ability to increase the growth rate of yeast cells expressing the fer cDNA. 30 substances from the sublibrary were found that increased the growth rate of the treated, Fer expressing cells to a level that was at least twice that of the growth rate observed in untreated, Fer expressing cells. These 30 substances were selected for repeat screening. FIG. 15 shows the results of the repeat screening of the 30 substances. It is seen that yeast cells expressing the HA-Fer kinase (pAES 426 HA-Fer) 49.5 hours after inoculation, grew less than yeast cells expressing the plasmid vector alone (pAES 426), and grew less than yeast expressing the mutant kinase HA-Fer Y715F (pAES 426 HA-Fer Y715F). In order to verify that the growth medium was free of contaminants, the medium was incubated in the absence of inoculated yeast cells (Ynb-Ura). In other wells, yeast cells expressing the HA-Fer (pAES 426 HA-Fer) were inoculated and at the time of inoculation a substance from the sublibrary, was added. As can be seen, there are substances having a molecular weight of up to 1000 Dalton, such as the substance referred to as “C9”, that are capable of restoring growth in the HA-Fer expressing cells to a level similar to that in the controls mentioned above. Other substances, such as the substance referred to as “D9”, did not significantly increase the growth rate.

The screening test described above in reference to FIG. 15 can also be carried out in the yeast strain BY4741-3702, having the following genotype: MATa, h is 3, leu 2, met 15, ura 3, PTC1D. This screening test can also be carried out in the yeast strain BY4741-3927, having the genotype-MATa, h is 3, leu 2, met 15, ura 3, PTPD.

Seven compounds that appeared positive in the repeated screening assay described above in reference to FIG. 15 were further tested for their ability to reverse, at least partially, the effect of Fer in the Saccharomyces cerevisiae strain 3702. In order to examine whether the alleviation of the inhibition caused by these compounds on these cells is dose dependent, compounds were added to yeast cultures in 10, 20 and 40 μM concentrations.

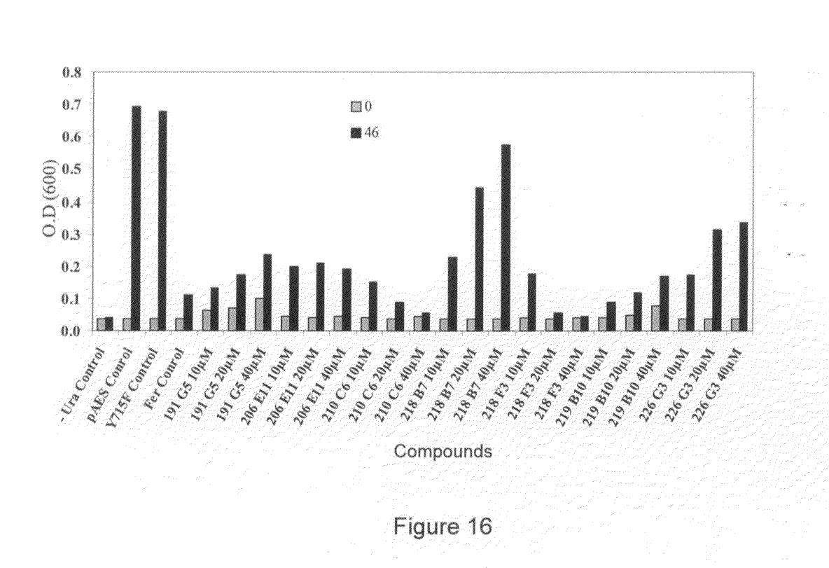

Referring now to FIG. 16, from left side of the histogram to the right side, bars represent growth level of the following: growth medium containing DMSO without yeast cells (Ura control), yeast transformed with the empty pAES vector in medium containing 2% (v/v) DMSO, yeast transformed with the pAES-HAFerY715F plasmid in medium containing 2% DMSO, yeast transformed with the pAES-HAFer in medium containing DMSO. All other bars represent yeast transformed with the HA-Fer in medium containing the indicted substance at the indicated concentration in DMSO.

Most of the compounds showed only a minor effect on the yeast growth rate after 46 hours, in comparison to yeasts incubated in the presence of DMSO alone. However, one compound referred to as “218B7”, exhibited a significant dose dependent growth restoring effect on the HA-Fer expressing cells.

To further test whether the compound 218B7, which affects the activity of Fer in yeast cells, can also affect the growth profile of cancer cells which express Fer, the compound was added to the growth media of several malignant cell lines which express Fer.

Cells from several malignant cell lines were seeded in 96 wells microplates and were left to grow untreated overnight. The compound 218B7 dissolved in DMSO was then added to each well to a final concentration of 80 μM. The concentration of DMSO in each well was 0.4% v/v. Untreated cells and cells subjected to 0.4% DMSO alone, served as controls. The number of viable cells in each well was determined 96 hours after compound addition, using the XTT test (Berridge, M. V. et al, 2005).

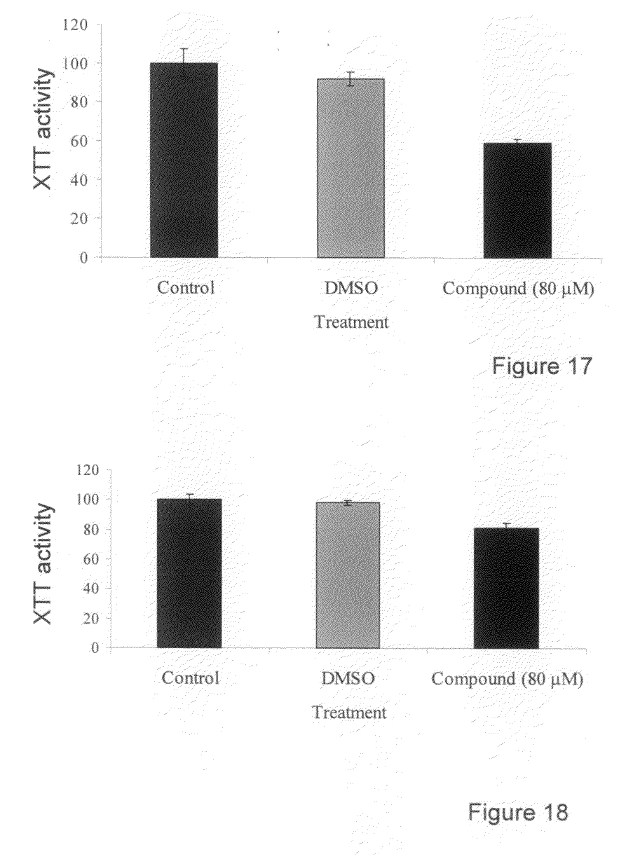

Two prostate cancer cell lines were examined the PC3 cell line (FIG. 17) and the DU 145 cell line (FIG. 18). In FIG. 17 it can be seen that the number of to viable prostate carcinoma PC3 cells in 218B7 treated cultures, was significantly reduced when compared to untreated controls or DMSO treated cells. The carcinoma cell—DU 145 was only slightly affected by the 218B7 compound (FIG. 18). In the histograms shown in FIGS. 17 to 19, the bars represent the average OD at 600 nm with level of significance p<0.0005 (student's t-test compared to treatment with DMSO alone). The small bars indicate standard deviation (n=8).

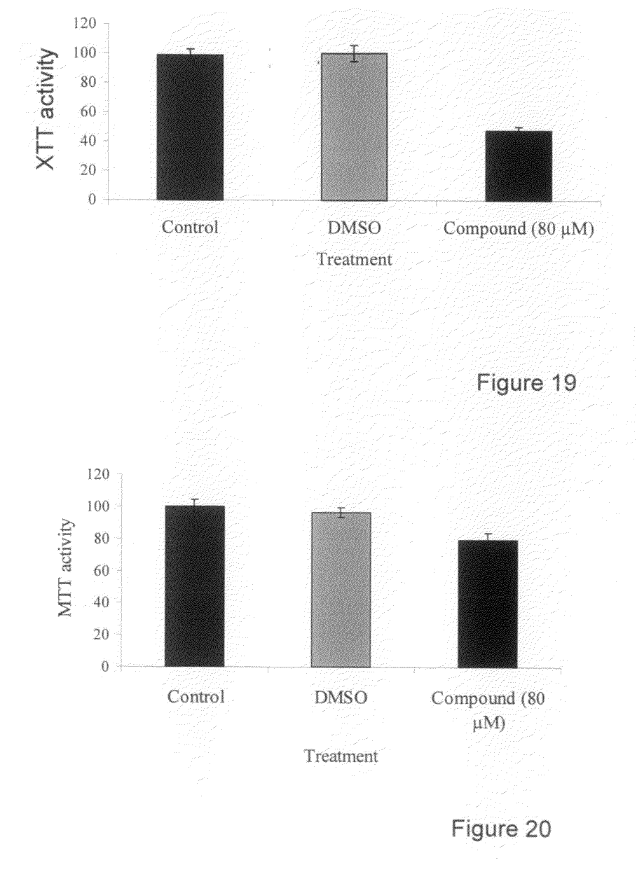

The effect of the 218B7 was also tested on two breast cancer cell lines MDA-MB231 and MCF-7. As can be seen in FIG. 19, MDA-MB-231 cells which originate from an aggressive metastatic breast cancer, suffered reduction of about 50% in the number of viable cells after being exposed to the 218B7 compound. The MCF-7 cells, which originate from a less aggressive breast cancer tumor, exhibited a 20% decrease in the number of viable cells after being treated with 218B7 (FIG. 20).

Without wishing to be bound by a particular theory, it its believed that the proliferation arrest of Fer depleted PC3 and MDA-MB-231 cells results from a sustained suppressive activity of pRB (Reed, 1997; Yamasaki, 2003), whose phosphorylation level (Cheng et al., 1999) was significantly reduced in the treated cells. Hypophosphorylation of pRB reflected the concomitant dephosphorylation of both CDK4 (Ser795 and Ser780) and CDK2 sites (Ser807, Ser811 and Thr821) in the Fer deprived PC3 cells. However, depletion of either one of these CDKs alone did not significantly affect the phosphorylation state of the pRB target sites. Moreover, simultaneous knock-down of these two key cell-cycle regulators in PC3 cells led to the dephosphorylation of Ser795 in pRB, but did not profoundly affect Thr821 which is a preferential target for CDK2 (Zarkowska and Mittnacht, 1997).

PP1 is a family of serine/threonine phosphatases which regulate a wide variety of cellular processes. Among these, PP1 activates pRB by removing neutralizing phosphate groups from key regulatory moieties in that tumor suppressing protein (Alberts et al., 1993; Durfee et al., 1993; Ludlow et al., 1993). Indeed, knock-down of Fer led to the dephosphorylation and activation of PP1α in one of the PP1 variants that were shown to dephosphorylate and activate pRB (Berndt et al., 1997). The pRB-associated PP1α pool is inactivated by CDK mediated phosphorylation on Thr320 (Liu et al., 1999) and depletion of Fer led to the hypophosphorylation of PP1α on that regulatory site. Conversely, over-expression of Fer increased the phosphorylation of PP1α on Thr320. Thus, Fer sustains the phosphorylation level of PP1α on Thr320, and thereby supports the down-regulation of that phosphatase. The ability of Fer to suppress the activity of PP1α was corroborated by the decreased in vitro phosphatase activity of PP1α from Fer over-expressing cells. This down-regulation of PP1α contributes most probably to the neutralization of the pRB suppressive activity, during G1 progression in malignant cells. Fer harbors two PP1 docking motifs in its kinase domain which most probably mediate the interaction of this kinase with PP1α in vivo. In accordance with this notion, mutations in these two PP1 binding motifs turned Fer into a dominant negative mutant that led to a decrease in the phosphorylation level of pRB. Thus, the interaction between Fer and PP1 is linked to the phosphorylation state of pRB and consequently to its cell-cycle regulatory activity. The dominant negative like effect of the HA-FerF606A/F649A mutant might be attributed to the ability of an exogenous dominant negative Fer to form hetero-oligomers (Craig et al., 2001) with the endogenous Fer and to interfere with its activity (Orlovsky et al., 2000). The association of Fer with PP1α might enhance the CDKs dependent phosphorylation of PP1α on Thr320. Alternatively, Fer could attenuate the auto or trans-dephosphorylation of PP1α ori Thr320 (Dohadwala et al., 1994). It should be noted that tyrosine phosphorylated PP1α was not detected in Fer expressing cells (data not shown), suggesting that the regulatory effect of Fer on PP1α is kinase activity independent, and may result from the physical interaction between these two proteins. Similarly, Fer binds ERK1/2 and maintains the phosphorylation states of these kinases, independently of the Fer tyrosine kinase activity (Salem et al., 2005).

1-24. (canceled)

25. A method for determining whether a substance is capable of altering or inhibiting an effect of a Fer tyrosine kinase, comprising:
(a) providing one or more cells expressing an exogenous fer gene or fer cDNA, wherein expression of the fer gene or fer cDNA in the cells leads to a decrease in proliferation rate of the cells in comparison to cells of the same type as the fer gene or fer cDNA expressing cells but not expressing the fer gene or fer cDNA;
(b) exposing the fer gene or fer cDNA expressing cells to the substance;
(c) measuring a rate of proliferation of the exposed fer gene or fer cDNA expressing cells and a proliferation rate of control fer gene or fer cDNA expressing cells not presented with the substance;
a proliferation rate of the exposed cells greater than the proliferation of the unexposed cells being indicative that the substance is capable of altering or inhibiting an effect of a Fer tyrosine kinase.

26. The method according to claim 25 wherein the fer gene or fer cDNA expressing cells first cell is a yeast cell.

27. The method according to claim 26 wherein the yeast cell is a Saccharomyces cerevisiae yeast cell.

28. The method according to claim 27 wherein the yeast cell is a Saccharomyces cerevisiae strain SP1 yeast cell.

29. The method according to claim 27 wherein the yeast cell is a Saccharomyces cerevisiae strain BY4741-3702 yeast cell.

30. The method according to claim 27 wherein the yeast cell is a Saccharomyces cerevisiae strain BY4741-3927 yeast cell.

31. One or more fer gene or fer cDNA expressing cells for use in the method of claim 25.

32. The one or more fer gene or fer cDNA expressing cells according to claim 31 wherein the one or more fer gene or fer cDNA expressing cells first cell is a yeast cell.

33. The one or more fer gene or fer cDNA expressing cells according to claim 32 wherein the yeast cell is a Saccharomyces cerevisiae yeast cell.

34. The one or more fer gene or fer cDNA expressing cells according to claim 33 wherein the yeast cell is a Saccharomyces cerevisiae strain SP1 yeast cell.

35. The one or more fer gene or fer cDNA expressing cells according to claim 33 wherein the yeast cell is a Saccharomyces cerevisiae strain BY4741-3702 yeast cell.

36. The one or more fer gene or fer cDNA expressing cells according to claim 33 wherein the yeast cell is a Saccharomyces cerevisiae strain BY4741-3927 yeast cell.

37. A method for determining whether a substance is capable of altering expression of a fer tyrosine kinase gene or a fer cDNA, comprising:
(a) providing one or more cells expressing an exogenous fer gene or fer cDNA, wherein expression of the fer gene or fer cDNA in the cells leads to a decrease in proliferation rate of the cells in comparison to cells of the same type as the fer gene or fer cDNA expressing cells but not expressing the fer gene or fer cDNA;
(b) exposing the fer gene or fer cDNA expressing cells to the substance;
(c) measuring a rate of proliferation of the exposed fer gene or fer cDNA expressing cells and a proliferation rate of control fer gene or fer cDNA expressing cells not presented with the substance;
a proliferation rate of the exposed cells greater than the proliferation of the unexposed cells being indicative that the substance is capable of altering expression of a fer tyrosine kinase gene or a fer cDNA.

38. The method according to claim 37 wherein the fer gene or fer cDNA expressing cells first cell is a yeast cell.

39. The method according to claim 38 wherein the yeast cell is a Saccharomyces cerevisiae yeast cell.

40. The method according to claim 39 wherein the yeast cell is a Saccharomyces cerevisiae strain SP1 yeast cell.

41. The method according to claim 39 wherein the yeast cell is a Saccharomyces cerevisiae strain BY4741-3702 yeast cell.

42. The method according to claim 39 wherein the yeast cell is a Saccharomyces cerevisiae strain BY4741-3927 yeast cell.

43. One or more fer gene or fer cDNA expressing cells for use in the method of claim 37.

44. The one or more fer gene or fer cDNA expressing cells according to claim 43 wherein the one or more fer gene or fer cDNA expressing cells first cell is a yeast cell.

45. The one or more fer gene or fer cDNA expressing cells according to claim 44 wherein the yeast cell is a Saccharomyces cerevisiae yeast cell.

46. The one or more fer gene or fer cDNA expressing cells according to claim 45 wherein the yeast cell is a Saccharomyces cerevisiae strain SP1 yeast cell.

47. The one or more fer gene or fer cDNA expressing cells according to claim 45 wherein the yeast cell is a Saccharomyces cerevisiae strain BY4741-3702 yeast cell.

48. The one or more fer gene or fer cDNA expressing cells according to claim 45 wherein the yeast cell is a Saccharomyces cerevisiae strain BY4741-3927 yeast cell.
